SEQUENCE LISTING (1 sequence)

SEQ ID NO: 1 - protein, 29 aa, synthetic construct
  REGION 1..29: note = Synthetic Peptide
  MOD_RES 2: note = Aib
  SITE 16: note = Glu side chain lactam linkage to Lys
  SITE 16..20: note = cyclic
  CHAIN 17: note = Z17CO2H
  SITE 20: note = Lys side chain lactam linkage to Glu
HXQGTFTSDYSKYLDEKAAKEFIQWLLQT

(19) United States
(12) Patent Application Publication
(10) Pub. No.: US 2025/0367263 A9
(43) Pub. Date: Dec. 4, 2025

(54) Therapeutic Regimens and Methods for Treatment of Cardiovascular Risk Factors using a GLP-1R and GCGR Agonist

(71) Applicant: Spitfire Pharma LLC, Gaithersburg, MD (US)

(72) Inventors: John Joseph Suschak, III, Damascus, MD (US); Bertrand Victor Gilbert Georges, London (GB); Sarah K. Browne, Gaithersburg, MD (US); Matthew Scott Harris, Gaithersburg, MD (US); M. Scot Roberts, Myersville, MD (US); Maynara Lucca Andrade, Gaithersburg, MD (US)

(73) Assignee: Spitfire Pharma LLC, Gaithersburg, MD (US)

(21) Appl. No.: 18/503,065

(22) Filed: Nov. 6, 2023

Related U.S. Application Data
(60) Provisional application No. 63/422,983, filed on Nov. 5, 2022.
(60) Provisional application No. 63/490,465, filed on Mar. 15, 2023.

(51) Int. Cl. A61K 38/26; A61P 3/04; A61P 3/06; A61P 9/00

(52) U.S. Cl. CPC A61K 38/26; A61P 3/04; A61P 3/06; A61P 9/00

(57) ABSTRACT

This disclosure relates to the once weekly dosing regimen of a dual GLP-1R and GCGR agonist, formulations, and methods of using the same for treatment of cardiovascular disease/disorder and/or reducing risk factors thereof by inducing reduction of pathogenic serum lipid mediators in a human being, wherein the human may or may not have other comorbidities such as obesity and/or type 2 diabetes. The dual GLP-1R and GCGR agonist includes the peptide product of SEQ ID NO. 1 (pemvidutide).

### RELATED APPLICATIONS

This application claims priority to Ser. No. 63/422,983 filed on 5 Nov. 2022 and to Ser. No. 63/490,491 filed on 15 Mar. 2023, each of which is incorporated into this application in its entirety.

### SEQUENCE LISTING

The instant application contains a Sequence Listing which has been submitted electronically in ASCII format via the USPTO Patent Center and hereby incorporated by reference in its entirety. Said ASCII copy, created on 15 Apr. 2024, is named MED013US1_Corr.xml and is 4293 bytes in size.

### FIELD OF THE DISCLOSURE

This disclosure relates to the use of the GLP-1R and GCGR agonist pemvidutide (ALT-801), a composition comprising SEQ ID NO.: 1) in certain dosing regimens for the treatment of obesity comorbidities, including cardiovascular (CV) disease/risk factors via reduction of pathogenic plasma lipid mediators.

### BACKGROUND OF THE DISCLOSURE

The increasing prevalence of cardiovascular (CV) disease and CV disease mortality in various human populations, including overweight and/or obese individuals, is a world health crisis of epidemic proportions that is a major contributor to patient morbidity and mortality as well as a major economic burden. For instance, obesity (e.g., persons having a body mass index (BMI, BMI kg/m²) of greater than 25 (overweight) or 30 (obese)) is a rapidly increasing problem worldwide and currently more than 65% of adults in the U.S. are overweight. And more than 80% of patients with heart failure with preserved ejection fraction (HFpEF) are overweight or obese (Kitzman, et al. Effect of caloric restriction or aerobic exercise training on peak oxygen consumption and quality of life in obese older patients with heart failure with preserved ejection fraction: A randomized clinical trial. JAMA. 2016; 315(1):36-46.) Weight loss of 7% has been shown to increase exercise tolerance and improve other measures of diastolic heart function in a study of 100 older patients (67+/−5 years) with obesity and clinically stable HFpEF (Id.; https://www.rethinkobesity.com/disease-progression/comorbidities-of-obesity.html). A majority of persons considered overweight or obese have limited options for available US Food and Drug Administration (FDA)-approved pharmacologic drugs for inducing weight loss, therapy has largely been based on lifestyle interventions directed at achieving weight loss. However, it is difficult to attain and maintain long-term weight loss with lifestyle changes alone.

Glucagon-like peptide-1 receptor agonists (GLP-1RA) are associated with modest degrees of weight loss at approved doses, and these agents have emerged as a treatment option for patients that are overweight. GLP-1RAs exert central effects on appetite and food intake, while GCGR agonists (GCGRAs) drive increased energy expenditure and lipid oxidation in animal models and humans. The effects of GCGRAs and GLP-1RA have been shown to be synergistic in driving greater degrees of weight loss compared to a GLP-1RA alone. GCGRAs also enhance lipolysis and suppress liver fat synthesis, providing an additional pathway for obesity-related conditions such as liver fat reduction, and/or resolution or improvement of non-alcoholic fatty liver disease (NAFLD) or non-alcoholic steatohepatitis (NASH), both conditions associated with being overweight, and especially obese. However, it has not yet been shown that such agonists have any effect on CV risk factors such a serum lipid levels or ratios.

Dual agonists combine GCGRAs with GLP-1RA in the same molecule. In obese non-human primates, chronic administration of a GLP-1R/GCGR dual agonist reduced body weight and improved glucose tolerance to a greater degree compared to a GLP-1RA mono-agonist. Clinical studies of cotadutide, a GLP-1/GCGR dual agonist with a 5:1 bias of GLP-1 to glucagon activity, demonstrated an impressive 39% reduction in liver fat content in just 6 weeks and greater improvement in NASH-related alanine aminotransferase (ALT) reduction than liraglutide alone. However, the degree of weight loss over 26 weeks of cotadutide administration was comparable to liraglutide (5.4% vs. 5.5%), suggesting that the 5:1 ratio was acceptable for liver fat reduction but suboptimal for weight reduction. Balanced (1:1) agonism has been shown to be associated with greater weight loss and metabolic effects than biased ratios that favor one agonist over the other. A recent study with JNJ 64565111, a balanced dual agonist, achieved an impressive 8% reduction in body weight in just 12 weeks (NCT03586830). It is unclear, however, whether the observed reduction in body weight has been associated with any CV disease-related factors (e.g., high cholesterol, triglycerides, LDL and VLDL particle concentration and/or diameter, and other related parameters).

Unfortunately, GLP-1Ras, and GLP-1R and GLP-1 based dual receptor agonists with bias towards GLP-1, have been associated with high rates of nausea, vomiting and diarrhea. These agents must also be titrated over prolonged periods to reduce side effects, and agents with improved tolerability and dosing regimens are needed. Accordingly, there remains a need for convenient dosing (e.g., weekly instead of daily) with a therapeutic dose to prevent and/or treat CV disease-related conditions that does not need to be titrated for long periods of time (e.g., more than 4 weeks) to reach a therapeutic level in the absence of gastrointestinal side effects. The compounds and methods presented herein provide a solution to such problems, and in particular are shown to have beneficial effects on CV disease-related factors.

### SUMMARY OF THE DISCLOSURE

Described herein are dual agonist peptides and products thereof (e.g., formulations) and uses of the same for treating cardiovascular (CV) related diseases (e.g., disorders) associated with the function of glucagon-like peptide 1 receptor (GLP-1R) and glucagon receptor (GCGR) to induce beneficial effects on CV disease-related factors such as high cholesterol, triglycerides, LDL and/or VLDL particle concentration and/or diameter, and/or other related parameters (e.g., serum phosphatidylethanolamines, phosphatidylcholines, lysophosphatidylethanolamines, sphingolipids and/or lysophosphatidylcholines). In certain embodiments the methods reduce, prevent, and/or normalize CV disease-related factors, wherein the method comprises administering pemvidutide once weekly in an amount from at least 1.2 mg up to 2.4 mg to the human being in need thereof. In embodiments, the body weight of the human being is reduced by at least 3%, or by at least 4% from baseline at week 12, wherein CV disease-related conditions such as high cholesterol, triglycerides, LDL and/or VLDL particle concentration and/or diameter, and/or other related parameters are also reduced or normalized (e.g., preferably reduced toward lower and/or normal levels.

In certain embodiments, the human being is overweight, obese, and/or suffers from type 2 diabetes. In alternative embodiments, the human being does not suffer from type 2 diabetes. In embodiments, the human being has a body mass index (BMI kg/m²) of at least 25, 27, 30, or greater. In certain embodiments, the administration of pemvidutide as disclosed herein results in the weight loss and/or a reduction in waistline measurements. In preferred embodiments, the CV disease-related conditions, including but not limited to high cholesterol, triglycerides, LDL and/or VLDL particle concentration and/or diameter, weight loss and/or reduction in waistline measurement, is significant as measured against an untreated or control (e.g., vehicle-treated) individual and/or population. In embodiments, the pemvidutide is administered once weekly in an amount of 1.2 mg, or once weekly in an amount of 2.4 mg. In certain embodiments, a steady state dose is achieved after a dose escalating phase having a duration of about 2 weeks, about 3 weeks or about 4 weeks. Some preferred embodiments are summarized in the aspects presented below. Other aspects of this disclosure are also contemplated as will be understood from the same by those of ordinary skill in the art.

### BRIEF DESCRIPTION OF THE DRAWINGS

FIG. 1 illustrates metabolite extraction, UHPLC-MS analysis, and data processing.

FIG. 2 shows serum lipoprotein changes analyzed by 2D-NMR in obese/overweight subjects treated with pemvidutide 1.2 mg (n=6), 1.8 mg (n=9), 2.4 mg (n=9) or placebo (n=10). Day 43 vs. Day −1, Day 84 vs. Day −1. The color code represents the log 2 (robust fold-change) with blue colors denoting reduced lipoproteins (negative fold-changes) and red colors denoting increased lipoproteins (positive fold-changes). See Example 1.

FIG. 3 shows the lipidomic signatures in obese/overweight subjects treated with pemvidutide 1.2 mg (n=6), 1.8 mg (n=9), 2.4 mg (n=9) or placebo (n=10) comparing changes at Day 84 vs Day −1. Results are presented as log 2 pairwise fold change with blue colors denoting reduced metabolites (negative fold-changes) and red colors denoting increased metabolites (positive fold-changes). Grey/black bars indicate significant p-values of Wilcoxon test (light grey, p<0,05; dark grey, p<0.01; black, p<0.001). (Definitions: PE=Phosphatidylethanolamine; PC=Phosphatidylcholine; PI=Phosphatidylinositol). Pemvidutide treatment significantly reduced serum lipid levels, especially glycerolipids, glycerophospholipids and sphingolipids within 12 weeks of treatment.

FIG. 4 illustrates volcano plots of changes in atherogenic lipid species following pemvidutide treatment at 1.8 mg dose. Y-axis is Log₁₀ of the statistical changes based on Student's T test with horizontal dotted line corresponding to p value threshold of 0.01. X-axis represents the Log₂ fold-change with dotted lines corresponding to arbitrary change threshold of ±0.75. Pemvidutide treatment significantly reduced pro-atherogenic lyso-PC levels, suggesting a potential for decreased oxidized LDL. Pemvidutide treatment significantly reduced atherosclerotic plaque forming lyso-phosphatidylcholine (PC) and phosphatidyl-ethanolamine (PE) glycerophospholipids levels

FIG. 5A illustrates significant weight loss induced by 12 weeks of weekly pemvidutide administration as compared to placebo.

FIG. 5B illustrates greater than or equal to 5% or 10% weight loss induced by 12 weeks of weekly pemvidutide administration as compared to placebo.

FIG. 6 shows baseline characteristics of study participants in Phase II study. See Example 2.

FIG. 7 shows percentage of subjects in each group achieving ≥5%, ≥10% or ≥15% body weight loss expressed as percentage of baseline body weight after 24-week treatment with Pemvidutide at 1.2 mg, 1.8 mg and 2.4 mg doses and placebo.

FIG. 8 show systolic and diastolic blood pressure expressed as mean percentage change from baseline (FIG. 8A) and heart rate expressed as mean percentage change from baseline (FIG. 8B) after 24-week treatment with Pemvidutide at 1.2 mg, 1.8 mg and 2.4 mg doses and placebo.

FIG. 9 shows serum lipids at week 24 as compared to baseline for all doses of Pemvidutide administered (1.2 mg, 1.8 mg, and 2.4 mg).

FIG. 10 shows a significant reduction in waist circumference at week 24 for all doses of Pemvidutide administered (1.2 mg, 1.8 mg, and 2.4 mg per week). See Example 2.

FIG. 11 shows the effect of pemvidutide and vehicle on % of weight loss (FIG. 11A), total cholesterol (FIG. 11B), LDL (FIG. 11C), HDL (FIG. 11D) and triglycerides (FIG. 11E) measured in diet-induced obese hamsters. See Example 3.

FIG. 12 shows the effect of pemvidutide and vehicle on liver weight (FIG. 12A), liver fatty acid (FIG. 12B), liver triglycerides (FIG. 12C) and liver cholesterol (FIG. 12D) measured in diet-induced obese hamsters 72 hours after injection of labelled macrophages. See Example 3.

FIG. 13 shows the effect of pemvidutide and vehicle on feces weight as measured in grams) FIG. 13A; % injected dose in fecal cholesterol per gram of feces (FIG. 13B); % injected dose in fecal bile acid per gram of feces (FIG. 13C); % injected dose in fecal cholesterol per total feces (FIG. 13D); and, % injected dose in fecal bile acids per total feces (FIG. 13E) measured in diet-induced obese hamsters treated after injection of labelled macrophages. See Example 3.

FIG. 14 shows the effect of pemvidutide and vehicle on % injected dose in plasma cholesterol in diet-induced obese hamsters 72 hours after injection of labelled macrophages. See Example 3.

FIG. 15 shows the effect of pemvidutide on the liver expression of CYP7A, ABCA1, ABCG1, ABCG5, ABCG8, SRB1, LDL-R or SREBP1c compared to semaglutide or vehicle. See Example 4.

### DETAILED DESCRIPTION OF THE DISCLOSURE

This disclosure relates to a dual agonist peptide(s) as well as pharmaceutical dosage formulations comprising, and methods for using, the same. The dual agonist peptides have affinity for, and in preferred embodiments about equal affinity for, glucagon-like peptide 1 receptor (GLP-1R) and glucagon receptor (GCGR), as may be determined using a cellular assay. In preferred embodiments, this disclosure provides methods for using pemvidutide (e.g., preferably pharmaceutical dosage formulation comprising SEQ ID NO: 1) that upon administration to a human being prevents and/or treats cardiovascular (CV) disease and/or CV disease-related conditions, symptoms, causes, and/or risks thereof. In some embodiments, the human being is overweight, obese (e.g., a body mass index of at least about 25), and may or may not suffer from type 2 diabetes. In preferred embodiments, the administration of pemvidutide as disclosed herein results in the weight loss and/or a reduction in waistline (“waist circumference”) measurements. In preferred embodiments, the CV disease-related conditions, including but not limited to high cholesterol, triglycerides, LDL and/or VLDL particle concentration and/or diameter, weight loss and/or reduction in waistline measurement, is significant as measured against an untreated or control (e.g., vehicle-treated) individual and/or population. In embodiments, the pemvidutide is administered once weekly in an amount of 1.2 mg, or once weekly in an amount of 2.4 mg. In certain embodiments, a steady state dose is achieved after a dose escalating phase having a duration of about 2 weeks, about 3 weeks or about 4 weeks. In some embodiments, this disclosure provides pharmaceutical dosage formulations configured to prevent and/or reduce conditions and/or parameters associated with CV disease-related conditions, including but not limited to reducing and/or normalizing serum cholesterol, triglyceride, VLDL/LDL particle concentration, and/or other parameters (e.g., scrum phosphatidylethanolamines, phosphatidylcholines, lysophosphatidylethanolamines, sphingolipids and/or lysophosphatidylcholines) in the human being. In some preferred embodiments, the methods include inducing the loss of body weight in the human being. In some preferred embodiments, the CV diseases include but are not limited to heart failure with preserved ejection fraction (HFpEF), coronary heart disease, cerebrovascular disease, peripheral arterial disease, rheumatic heart disease, congenital heart disease, and/or deep vein thrombosis and pulmonary embolism. In preferred embodiments, the method comprises administering pemvidutide once weekly in an amount from at least about any of 1.2 mg, 1.8 mg, or 2.4 mg pemvidutide to the human being in need thereof. In certain embodiments, the method comprises administering pemvidutide once weekly in an amount of 1.8 mg to the human being in need thereof for treatment of CV disease/disorder by inducing a reduction of pathogenic serum lipid mediators. As used herein, “pathogenic” serum lipid mediators, included but not limited to the following reactive lipid species: malondialdehyde (MDA), isolevuglandins (IsoLG), methyglyoxal (MGO), 4-oxononenal (ONE), and 4-hydroxynonenal (HNE). Oxidized phospholipids include 1-palmitoyl-2-oxovaleroyl-sn-glycero-3-phosphorylcholine (POVPC), 1-O-alkyl-2-azelaoyl-sn-glycero-3-phophorylcholine (azPAF), 1-(Palmitoyl)-2-(5-keto-6-octenc-dioyl) phosphatidylcholine (KOdiA-PC), 1-palmitoyl-2-F2-isoprostane-sn-glycero-3-phosphocholine (F2IsoP-PC), and 1-palmitoyl-2-(5,6)-epoxyisoprostane E2-sn-glycero-3-phosphocholine (PEIPC).

Pemvidutide, also referred to herein as ALT-801, is a composition comprising a synthetic peptide (SEQ ID NO: 1) composed of naturally occurring amino acids and is a chimeric analog of the two native hormones GLP-1 and glucagon, with predominantly glucagon residues in the N-terminus and GLP-1 residues in the C-terminus. See U.S. Pat. No. 9,856,306, incorporated herein by reference. ALT-801 also incorporates one nonproteinogenic amino acid, 2-aminoisobutyric acid, an amino acid side chain amide linkage (lactam bridge), and a surfactant side chain composed of a glucuronic acid linked to an octadecanoic fatty acid side chain. The surfactant side chain appears to slow entry in the circulation and may form micelles after subcutaneous (SC) injection. The lower maximal concentration (Cₘₐₓ) associated with slower entry may result in fewer GI side effects and better tolerability. This latter feature also enhances binding to plasma proteins and improves the metabolic stability, extending the half-life (t_{1/2}). The design of ALT-801 provides a co-agonist with equipotent (1:1) activity at both receptors of approximately 40 pM and 100% activity. Compositions comprising SEQ ID NO: 1 has been administered to human beings across a variety of doses found not to induce side effects such as nausea, vomiting, diarrhea, abdominal pain and/or constipation using standard techniques. See US Patent Publ. No. 2021/0290732 and PCT Publication No. WO 2022/125598, each incorporated herein by reference.

ALT-801 (SEQ ID NO: 1) has the following amino acid sequence:

¹His-²Aib-³Gln-⁴Gly-⁵Thr-⁶Phe-⁷Thr-⁸Ser-⁹Asp-¹⁰Tyr-
¹¹Ser-¹²Lys-¹³Tyr-¹⁴Leu-¹⁵Asp-¹⁶Glu*-¹⁷Lys^{#}-¹⁸Ala-¹⁹Ala-
²⁰Lys*-²¹Glu-²²Phe-²³Ile-²⁴Gln-²⁵Trp-²⁶Leu-²⁷Leu-²⁸Gln-
²⁹Thr-NH₂,

where * indicates a lactam bridge is formed between Glu16 and Lys 20, and 17Lys #indicates the attachment site for glucuronic acid C-18*(Z17CO2H) (“1-(17-carboxylheptadecyloxy)-beta-D-glucuronyl”). Illustrated differently, SEQ ID NO: 1 is a peptide amide consisting of 29 amino acid residues and a glucuronic acid/C₁₈ diacid moiety attached to ¹⁷Lys, in which the side-chains of ¹⁶Glu and ²⁰Lys forming an intramolecular cycle as shown below:

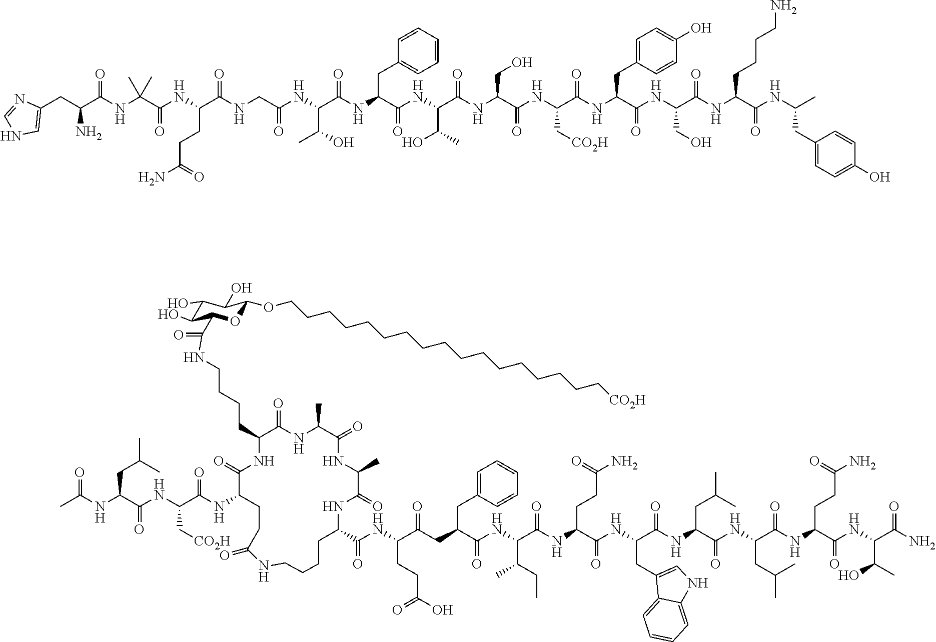

In embodiments provided herein is a pharmaceutical formulation of SEQ ID NO: 1 in an aqueous buffer solution, referred to herein as ALT-801. The dual agonist peptide products herein, including SEQ ID NO: 1, comprise an amino acid side chain amide linkage (lactam bridge), and a surfactant side chain composed of a glucuronic acid linked to a fatty acid side chain. The side chain, a surfactant comprised of a hydrophilic saccharide group covalently attached to the peptide via a linker amino acid, and a hydrophobic alkyl chain portion. In some embodiments, the dual agonist peptides can include one or more conservatively substituted amino acids as described herein. In preferred embodiments, SEQ ID NO: 1 can include one or more conservatively substituted amino acids, but preferably not at amino acid residues 16, 17, or 20.

A “peptide” (e.g., dual agonist peptide) comprises two or more natural or/and unnatural amino acid residues linked typically via peptide bonds. Such amino acids can include naturally occurring structural variants, naturally occurring non-proteinogenic amino acids, or/and synthetic non-naturally occurring analogs of natural amino acids. The terms “peptide” and “polypeptide” are used interchangeably herein. Peptides include short peptides (about 2-20 amino acids), medium-length peptides (about 21-50 amino acids) and long peptides (>about 50 amino acids, which can also be called “proteins”). In some embodiments, a peptide product comprises a surfactant moiety covalently and stably attached to a peptide of no more than about 50, 40 or 30 amino acids. Synthetic peptides can be synthesized using an automated peptide synthesizer, for example. Peptides can also be produced recombinantly in cells expressing nucleic acid sequences that encode the peptides. Conventional notation is used herein to portray peptide sequences: the left-hand end of a peptide sequence is the amino (N)-terminus, and the right-hand end of a peptide sequence is the carboxyl (C)-terminus. Standard one-letter and three-letter abbreviations for the common amino acids are used herein. Although the abbreviations used in the amino acid sequences disclosed herein represent L-amino acids unless otherwise designated as D- or DL- or the amino acid is achiral, the counterpart D-isomer generally can be used at any position (e.g., to resist proteolytic degradation). Abbreviations for other amino acids used herein include: Aib=a-aminoisobutyric acid (or 2-methylalanine or Ca-methylalanine); Xaa: any amino acid, typically specifically defined within a formula. Abbreviations for other amino acids that can be used as described herein include: Ac3c=1-aminocyclopropane-1-carboxylic acid; Ac4c=1-aminocyclobutane-1-carboxylic acid; Ac5c=1-aminocyclopentane-1-carboxylic acid; Ac6c=1-aminocyclohexane-1-carboxylic acid; Aib=alpha-aminoisobutyric acid (or 2-methylalanine or Calpha-methylalanine); Bip=3-(biphenyl-4-yl)alanine; Bip2Et=3-(2′-ethylbiphenyl-4-yl)alanine; Bip2EtMeO=3-(2′-ethyl-4′-methoxybiphenyl-4-yl)alanine; Cit=citrulline; Deg=2,2-diethylglycine; Dmt=(2,6-dimethyl)tyrosine; 2FPhe=(2-fluorophenyl)alanine; 2FMePhe or 2FaMePhe=Ca-methyl-(2-fluorophenyl)alanine; hArg=homoarginine; MeLys or aMeLys=Ca-methyllysine; MePhe or aMePhe=Ca-methylphenylalanine; MePro or aMePro=Ca-methylproline; Nall or Nal(1)=3-(1-naphthyl)alanine; Nal2 or Nal(2)=3-(2-naphthyl)alanine; Nle=norleucine; Om=ornithine; and Tmp=(2,4,6-trimethylphenyl)alanine; 1,2,3,4-tetrahydroisoquinoline-3-carboxylic acid (Tic) and a Tic-Phe dipeptide moiety with a reduced amide bond between the residues (designated as Tic-Ψ[CF12-NF1]-Ψ-Phe) have the following structures:

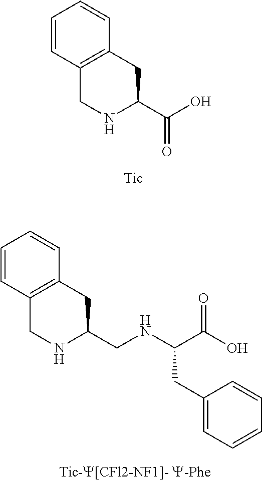 [SMILES: O=C(O)[C@@H]1Cc2ccccc2CN1 ; O=C(O)[C@H](Cc1ccccc1)NC[C@@H]1Cc2ccccc2CN1]

Unless specifically stated otherwise or the context clearly indicates otherwise, the disclosure encompasses any and all forms of a dual agonist peptide that may be produced, whether the dual agonist peptide is produced synthetically (e.g., using a peptide synthesizer) or by a cell (e.g., by recombinant production). Such forms of a dual agonist peptide can include one or more modifications that may be made during the course of synthetic or cellular production of the peptide, such as one or more post-translational modifications, whether or not the one or more modifications are deliberate. A dual agonist peptide can have the same type of modification at two or more different places, or/and can have two or more different types of modifications. Modifications that may be made during the course of synthetic or cellular production of a dual agonist peptide, including chemical and post-translational modifications, include without limitation glycosylation (e.g., N-linked glycosylation and O-linked glycosylation), lipidation, phosphorylation, sulfation, acetylation (e.g., acetylation of the N-terminus), amidation (e.g., amidation of the C-terminus), hydroxylation, methylation, formation of an intramolecular or intermolecular disulfide bond, formation of a lactam between two side chains, formation of pyroglutamate, and ubiquitination. A dual agonist peptide can have one or more modifications anywhere, such as the N-terminus, the C-terminus, one or more amino acid side chains, or the dual agonist peptide backbone, or any combination thereof. In some embodiments, a dual agonist peptide is acetylated at the N-terminus or/and has a carboxamide (—CONH₂) group at the C-terminus, which can increase the stability of the dual agonist peptide.

Potential modifications of a dual agonist peptide also include deletion of one or more amino acids, addition/insertion of one or more natural or/and unnatural amino acids, or substitution with one or more natural or/and unnatural amino acids, or any combination or all thereof. A substitution can be conservative or non-conservative. Such modifications may be deliberate, such as via site-directed mutagenesis or in the chemical synthesis of a dual agonist peptide, or may be accidental, such as via mutations arising in the host cell that produces the dual agonist peptide or via errors due to PCR amplification. An unnatural amino acid can have the same chemical structure as the counterpart natural amino acid but have the D stereochemistry, or it can have a different chemical structure and the D or L stereochemistry. Unnatural amino acids can be utilized, e.g., to promote a-helix formation or/and to increase the stability of the dual agonist peptide (e.g., to resist proteolytic degradation). A dual agonist peptide having one or more modifications relative to a reference dual agonist peptide may be called an “analog” or “variant” of the reference dual agonist peptide as appropriate. An “analog” typically retains one or more essential properties (e.g., receptor binding, activation of a receptor or enzyme, inhibition of a receptor or enzyme, or other biological activity) of the reference dual agonist peptide. A “variant” may or may not retain the biological activity of the reference dual agonist peptide, or/and may have a different biological activity. It is preferred that such a variant maintain its ability to act as an agonist of GLP-1R and GCGR, and in more preferred embodiments, has about equal affinity for GLP-1R and GCGR. In some embodiments, an analog or variant of a reference peptide has a different amino acid sequence than the reference dual agonist peptide.

The term “conservative substitution” refers to substitution of an amino acid in a dual agonist peptide with a functionally, structurally or chemically similar natural or unnatural amino acid. In certain embodiments, the following groups each contain natural amino acids that are conservative substitutions for one another: 1) Glycine (Gly/G), Alanine (Ala/A); 2) Isoleucine (Ile/I), Leucine (Leu/L), Methionine (Met/M), Valine (Val/V); 3) Phenylalanine (Phe/F), Tyrosine (Tyr/Y), Tryptophan (Trp/W); 4) Serine (Ser/S), Threonine (Thr/T), Cysteine (Cys/C); 5) Asparagine (Asn/N), Glutamine (Gln/Q); 6) Aspartic acid (Asp/D), Glutamic acid (Glu/E); and, 7) Arginine (Arg/R), Lysine (Lys/K), Histidine (His/H). In further embodiments, the following groups each contain natural amino acids that are conservative substitutions for one another: 1) non-polar: Ala, Val, Leu, Ile, Met, Pro (proline/P), Phe, Trp; 2) hydrophobic: Val, Leu, Ile, Phe, Trp; 3) aliphatic: Ala, Val, Leu, Ile; 4) aromatic: Phe, Tyr, Trp, His; 5) uncharged polar or hydrophilic: Gly, Ala, Pro, Ser, Thr, Cys, Asn, Gln, Tyr; 6) aliphatic hydroxyl- or sulfhydryl-containing: Ser, Thr, Cys; 7) amide-containing: Asn, Gln; 8) acidic: Asp, Glu; 9) basic: Lys, Arg, His; and, 10) small: Gly, Ala, Ser, Cys. In other embodiments, amino acids may be grouped as conservative substitutions as set out below: 1) hydrophobic: Val, Leu, Ile, Met, Phe, Trp; 2) aromatic: Phe, Tyr, Trp, His; 3) neutral hydrophilic: Gly, Ala, Pro, Ser, Thr, Cys, Asn, Gln; 4) acidic: Asp, Glu; 5) basic: Lys, Arg, His; and, 6) residues that influence backbone orientation: Pro.

Examples of unnatural or non-proteinogenic amino acids include without limitation alanine analogs (e.g., α-ethylGly [α-aminobutyric acid or Abu], α-n-propylGly [norvaline or Nva], α-tert-butylGly [Tbg], α-vinyl Gly [Vg or Vlg], α-allylGly [Alg], α-propargylGly [Prg], 3-cyclopropylAla [Cpa] and Aib), leucine analogs (e.g., nor-leucine, Nle), proline analogs (e.g., α-MePro), phenylalanine analogs (e.g., Phe(2-F), Phe(2-Me), Tmp, Bip, Bip(2′-Et-4′-OMe), Nal1, Nal2, Tic, α-MePhe, α-MePhe(2-F) and α-MePhe(2-Me)), tyrosine analogs (e.g., Dmt and α-MeTyr), serine analogs (e.g., homoserine [isothreonine or hSer]), glutamine analogs (e.g., Cit), arginine analogs (e.g., hArg, N,N′-g-dialkyl-hArg), lysine analogs (e.g, homolysine [hLys], Orn and α-MeLys), α, α-disubstituted amino acids (e.g., Aib, α, α-diethylGly [Deg], α-cyclohexylAla [2-Cha], Ac3c, Ac4c, Ac5c and Ac6c), and other unnatural amino acids disclosed in A. Santoprete et al., Pept. Sci., 17:270-280 (2011). α,α-Di-substituted amino acids can provide conformational restraint or/and α-helix stabilization. A reduced amide bond between two residues (as in, e.g., Tic-Ψ[CF12-NF1]-Ψ-Phe) increases protease resistance and may also, e.g., alter receptor binding. The disclosure encompasses all pharmaceutically acceptable salts of dual agonist peptides, including those with a positive net charge, those with a negative net charge, and those with no net charge.

An “alkyl” group refers to an aliphatic hydrocarbon group. An alkyl group can be saturated or unsaturated, and can be straight chain (linear), branched or cyclic. In some embodiments, an alkyl group is not cyclic. In some embodiments, an alkyl group contains 1-30, 6-30, 6-20 or 8-20 carbon atoms. A “substituted” alkyl group is substituted with one or more substituents. In some embodiments, the one or more substituents are independently selected from halogens, nitro, cyano, oxo, hydroxy, alkoxy, haloalkoxy, aryloxy, thiol, alkylthio, arylthio, alkylsulfoxide, arylsulfoxide, alkylsulfone, arylsulfone, amino, alkylamino, dialkylamino, arylamino, alkoyl, carboxyl, carboxylate, esters, amides, carbonates, carbamates, ureas, alkyl, haloalkyl, fluoroalkyl, aralkyl, alkyl chains containing an acyl group, heteroalkyl, heteroali-cyclic, aryl, alkoxyaryl, heteroaryl, hydrophobic natural compounds (e.g., steroids), and the like. In some embodiments, an alkyl group as a substituent is linear or branched Ci-C₆ alkyl, which can be called “lower alkyl”. Non-limiting examples of lower alkyl groups include methyl, ethyl, propyl (including n-propyl and isopropyl), butyl (including all isomeric forms, such as n-butyl, isobutyl, sec-butyl and/er/-butyl), pentyl (including all isomeric forms, such as n-pentyl), and hexyl (including all isomeric forms, such as n-hexyl). In some embodiments, an alkyl group is attached to the Na-atom of a residue (e.g., Tyr or Dmt) of a peptide. In certain embodiments, an N-alkyl group is straight or branched C1-C₁₀ alkyl, or aryl-substituted alkyl such as benzyl, phenylethyl or the like. One or two alkyl groups can be attached to the Na-atom of the N-terminal residue. In some embodiments, an alkyl group is a 1-alkyl group that is attached to the C-1 position of a saccharide (e.g., glucose) via a glycosidic bond (e.g., an O-, S-, N- or C-glycosidic bond). In some embodiments, such a 1-alkyl group is an unsubstituted or substituted C1-C30, C₆-C30, C₆-C₂₀ or C₈-C₂₀ alkyl group. In some embodiments, an alkyl group (e.g., a 1-alkyl group) is substituted with one or more (e.g., 2 or 3) groups independently selected from aryl, —OH, —OR¹, —SH, —SR¹, —NH₂, —NHR¹, —N(R¹)₂, oxo (═O), —C(═O)R², carboxyl (—CO₂H), carboxylate (—CO₂), —C(═O)OR¹, —OC(═O)R³, —C(═O)N(R¹)₂, —NR⁴C(═O)R³, —OC(═O)OR⁵, —OC(═O)N(R¹)₂, —NR⁴C(═O)OR⁵, and —NR⁴C(═O)N(R¹)₂, wherein: R¹ at each occurrence independently is hydrogen, alkyl or aryl, or both occurrences of R¹ and the nitrogen atom to which they are connected form a heterocyclyl or heteroaryl ring; R² at each occurrence independently is alkyl, heterocyclyl, aryl or heteroaryl; R³ at each occurrence independently is hydrogen, alkyl, heterocyclyl, aryl or heteroaryl; R⁴ at each occurrence independently is hydrogen or alkyl; and, R⁵ at each occurrence independently is alkyl or aryl. In some embodiments, an alkyl group (e.g., a 1-alkyl group) is internally or/and terminally substituted with a carboxyl/carboxylate group, an aryl group or an —O-aryl group. In certain embodiments, an alkyl group (e.g., a 1-alkyl group) is substituted with a carboxyl or carboxylate group at the distal end of the alkyl group. In further embodiments, an alkyl group (e.g., a 1-alkyl group) is substituted with an aryl group at the distal end of the alkyl group. In other embodiments, an alkyl group (e.g., a 1-alkyl group) is substituted with an —O-aryl group at the distal end of the alkyl group. The terms “halogen”, “halide” and “halo” refer to fluoride, chloride, bromide and iodide. The term “acyl” refers to —C(═O)R, where R is an aliphatic group that can be saturated or unsaturated, and can be linear, branched or cyclic. In certain embodiments, R contains 1-20, 1-10 or 1-6 carbon atoms. An acyl group can optionally be substituted with one or more groups, such as halogens, oxo, hydroxyl, alkoxy, thiol, alkylthio, amino, alkylamino, dialkylamino, cycloalkyl, aryl, acyl, carboxyl, esters, amides, hydrophobic natural compounds (e.g., steroids), and the like. The terms “heterocyclyl” and “heterocyclic” refer to a monocyclic non-aromatic group or a multicyclic group that contains at least one non-aromatic ring, wherein at least one non-aromatic ring contains one or more heteroatoms independently selected from O, N and S. The non-aromatic ring containing one or more heteroatoms may be attached or fused to one or more saturated, partially unsaturated or aromatic rings. In certain embodiments, a heterocyclyl or heterocyclic group has from 3 to 15, or 3 to 12, or 3 to 10, or 3 to 8, or 3 to 6 ring atoms. Heterocyclyl or heterocyclic groups include without limitation aziridinyl, azetidinyl, pyrrolidinyl, piperidinyl, morpholinyl, piperazinyl, azepanyl, azocanyl, oxiranyl, oxetanyl, tetrahydrofuranyl (oxolanyl), tetrahydropyranyl, oxepanyl and oxocanyl. The term “aryl” refers to a monocyclic aromatic hydrocarbon group or a multicyclic group that contains at least one aromatic hydrocarbon ring. In certain embodiments, an aryl group has from 6 to 15, or 6 to 12, or 6 to 10 ring atoms. Aryl groups include without limitation phenyl, naphthalenyl (naphthyl), fluorenyl, azulenyl, anthryl, phenanthryl, biphenyl and terphenyl. The aromatic hydrocarbon ring of an aryl group may be attached or fused to one or more saturated, partially unsaturated or aromatic rings—e.g., dihydronaphthyl, indenyl, indanyl and tetrahydronaphthyl (tetralinyl). An aryl group can optionally be substituted with one or more (e.g., 2 or 3) substituents independently selected from halogens (including —F and —Cl), cyano, nitro, hydroxyl, alkoxy, thiol, alkylthio, alkylsulfoxide, alkylsulfone, amino, alkylamino, dialkylamino, alkyl, haloalkyl (including fluoroalkyl such as trifluoromethyl), acyl, carboxyl, esters, amides, and the like. The term “heteroaryl” refers to a monocyclic aromatic group or a multicyclic group that contains at least one aromatic ring, wherein at least one aromatic ring contains one or more heteroatoms independently selected from O, N and S. The heteroaromatic ring may be attached or fused to one or more saturated, partially unsaturated or aromatic rings that may contain only carbon atoms or that may contain one or more heteroatoms. In certain embodiments, a heteroaryl group has from 5 to 15, or 5 to 12, or 5 to 10 ring atoms. Monocyclic heteroaryl groups include without limitation pyrrolyl, pyrazolyl, pyrazolinyl, imidazolyl, oxazolyl, isoxazolyl, thiazolyl, thiadiazolyl, isothiazolyl, furanyl, thienyl (thiophenyl), oxadiazolyl, triazolyl, tetrazolyl, pyridyl, pyridonyl, pyrazinyl, pyrimidinyl, pyridazinyl, pyridazinonyl and triazinyl. Non-limiting examples of bicyclic heteroaryl groups include indolyl, benzothiazolyl, benzothiadiazolyl, benzoxazolyl, benzisoxazolyl, benzothienyl (benzothiophenyl), quinolinyl, tetrahydroisoquinolinyl, isoquinolinyl, benzimidazolyl, benzotriazolyl, indolizinyl, benzofuranyl, isobenzofuranyl, chromonyl, coumarinyl, cinnolinyl, quinazolinyl, quinoxalinyl, indazolyl, naphthyridinyl, phthalazinyl, quinazolinyl, purinyl, pyrrol opyridinyl, furopyridinyl, thienopyridinyl, dihydroisoindolyl and tetrahydroquinolinyl.

In some embodiments, for instance, the dual agonist peptides can be associated with a saccharide, such as within a pharmaceutically acceptable composition or lyophilizate. Saccharides include monosaccharides, disaccharides and oligosaccharides (e.g., trisaccharides, tetrasaccharides and so on). A reducing saccharide exists in a ring form and an open-chain form in equilibrium, which generally favors the ring form. A functionalized saccharide of a surfactant moiety has a functional group suitable for forming a stable covalent bond with an amino acid of a dual agonist peptide.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition. In one embodiment, a pharmaceutically acceptable composition in which a dual agonist peptide can be formulated comprises polysorbate 20 (e.g., about 0.050% (w/w)); optionally methylparaben (e.g., about 0.300% (w/w)); arginine (about 0.348% (w/w)), and mannitol (e.g., about 4.260% (w/w)) in distilled (DI) water.

The term “therapeutically effective amount” refers to an amount of a compound that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that compound. The term “therapeutically effective amount” also refers to an amount of a compound that is sufficient to elicit the biological or medical response of a cell, tissue, organ or human which is sought by a medical doctor or clinician.

The terms “treat,” “treating” and “treatment” include alleviating, ameliorating, inhibiting the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk of developing and delaying the onset of a medical condition or one or more symptoms or complications associated with the condition. The term “medical conditions” (or “conditions” for brevity) includes diseases and disorders. The terms “diseases” and “disorders” are used interchangeably herein.

The disclosure also provides pharmaceutical compositions comprising a dual agonist peptide product described herein or a pharmaceutically acceptable salt thereof, and one or more pharmaceutically acceptable carriers or excipients. A pharmaceutical composition contains a therapeutically effective amount of a peptide product or an appropriate fraction thereof. A composition can optionally contain an additional therapeutic agent. In some embodiments, a peptide product is at least about 90%, 95% or 98% pure. Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents (e.g., saline, buffered saline (e.g., phosphate-buffered saline [PBS]) and isotonic solutions (e.g., Ringer's solution)), and organic solvents (e.g., dimethyl sulfoxide and alcohols [e.g., ethanol, glycerol and propylene glycol]). Except insofar as any conventional excipient or carrier is incompatible with a peptide product, the disclosure encompasses the use of conventional excipients and carriers in formulations containing a peptide product. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et ah, Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004).

In embodiments, a pharmaceutical formulation comprises a peptide product and about 0.02-0.075% (w/w) polysorbate 20, about 0.2-0.5% (w/w) arginine, about 3-6% (w/w) mannitol in sterile water (pH 7.7±0.1); optionally about 0.050% (w/w) polysorbate 20, about 0.348% (w/w) arginine, about 4.260% (w/w) mannitol in sterile water (pH 7.7±0.1). In certain embodiments, a present pharmaceutical formulation comprises SEQ ID NO: 1 and about 0.050% (w/w) polysorbate 20, about 0.348% (w/w) arginine, about 4.260% (w/w) mannitol in sterile water (pH 7.7±0.1). In certain embodiments, a present pharmaceutical formulation comprises SEQ ID NO: 1 and about 0.20% (w/w) polysorbate 20, about 0.348% (w/w) arginine, about 4.260% (w/w) mannitol in sterile water (pH 7.7±0.1). In certain embodiments, the pharmaceutical formulation comprises SEQ ID NO: 1 and is configured for subcutaneous (SC) administration of a weekly therapeutic dose.

An appropriate or suitable formulation can depend on various factors, such as the route of administration chosen. Potential routes of administration of a pharmaceutical composition comprising a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository). In certain embodiments, a present dual agonist peptide product is administered parenterally (e.g., subcutaneously, intravenously or intramuscularly). In other embodiments, a peptide product is administered by oral inhalation or nasal inhalation or insufflation. In some embodiments, the carrier is an aqueous-based carrier, such as in a parenteral (e.g., subcutaneous, intravenous or intramuscular) formulation. In other embodiments, the carrier is a nonaqueous-based carrier. In certain embodiments, the nonaqueous-based carrier is a hydrofluoroalkane (HFA) or HFA-like solvent that may comprise sub-micron anhydrous a-lactose or/and other excipients, such as in a formulation for administration by oral inhalation or nasal inhalation or insufflation.

In some embodiments, a peptide product is administered parenterally (e.g., subcutaneously, intravenously or intramuscularly) by injection. Parenteral administration bypasses the strongly acidic environment of the stomach, gastrointestinal (GI) absorption and first-pass metabolism. Excipients and carriers that can be used to prepare parenteral formulations include without limitation solvents (e.g., aqueous solvents such as water, saline, physiological saline, buffered saline [e.g., PBS], balanced salt solutions [e.g., Ringer's BSS] and aqueous dextrose solutions), isotonic/iso-osmotic agents (e.g., salts [e.g., NaCl, KCl and CaCl₂)] and sugars [e.g., sucrose]), buffering agents and pH adjusters (e.g., sodium dihydrogen phosphate [monobasic sodium phosphate]/di sodium hydrogen phosphate [dibasic sodium phosphate], citric acid/sodium citrate and L-histidine/L-histidine HCl), and emulsifiers (e.g., non-ionic surfactants such as polysorbates [e.g., polysorbate 20 and 80] and poloxamers [e.g., poloxamer 188]). Peptide formulations and delivery systems are discussed in, e.g., A. J. Banga, Therapeutic Peptides and Proteins: Formulation, Processing, and Delivery Systems, 3rd Ed., CRC Press (Boca Raton, Florida) (2015). The excipients can optionally include one or more substances that increase peptide stability, increase peptide solubility, inhibit peptide aggregation or reduce solution viscosity, or any combination or all thereof. Such substances include without limitation hydrophilic amino acids (e.g., arginine and histidine), polyols (e.g., myo-inositol, mannitol and sorbitol), saccharides (e.g., glucose (including D-glucose [dextrose]), lactose, sucrose and trehalose}, osmolytes (e.g., trehalose, taurine, amino acids [e.g., glycine, sarcosine, alanine, proline, serine, b-alanine and g-aminobutyric acid], and betaines [e.g., trimethylglycine and trimethylamine N-oxide]), and non-ionic surfactants (e.g., alkyl polyglycosides, ProTek® alkylsaccarides (e.g., a monosaccharide [e.g., glucose] or a disaccharide [e.g., maltose or sucrose] coupled to a long-chain fatty acid or a corresponding long-chain alcohol), and polypropylene glycol/polyethylene glycol block co-polymers (e.g., poloxamers [e.g., Pluronic™F-68], and Genapol® PF-10 and variants thereof). Because such substances increase peptide solubility, they can be used to increase peptide concentration in a formulation. Higher peptide concentration in a formulation is particularly advantageous for subcutaneous administration, which has a limited volume of bolus administration (e.g., <about 1.5 mL). In addition, such substances can be used to stabilize peptides during the preparation, storage and reconstitution of lyophilized peptides. An exemplary parenteral formulation comprises a peptide product, mannitol, methionine, sodium thioglycolate, polysorbate 20, a pH adjuster (e.g., NaOH or/and HCl) and de-ionized water. Excipients of parenteral formulations that would be suitable for use with the dual agonist peptides described herein (e.g., various combinations of excipients including NaCl and the like) are well-known and available to those of ordinary skill in the art.

For parenteral (e.g., subcutaneous, intravenous or intramuscular) administration, a sterile solution or suspension of a peptide product in an aqueous solvent containing one or more excipients can be prepared beforehand and can be provided in, e.g., a pre-filled syringe of a single-use pen or a pen with a dose counter. Alternatively, a peptide product can be dissolved or suspended in an aqueous solvent that can optionally contain one or more excipients prior to lyophilization (freeze-drying). Shortly prior to parenteral administration, the lyophilized peptide product stored in a suitable container (e.g., a vial) can be reconstituted with, e.g., sterile water that can optionally contain one or more excipients. In other embodiments, an agonist peptide product is administered intranasally. The nasal mucosa provides a big surface area, a porous endothelium, a highly vascular subepithelial layer and a high absorption rate, and hence allows for high bioavailability. An intranasal formulation can comprise a peptide product along with excipients, such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. An intranasal solution or suspension formulation can be administered to the nasal cavity by any suitable means, including but not limited to a dropper, a pipette, or spray using, e.g., a metering atomizing spray pump. Table 2 shows exemplary excipients of nasal-spray formulations.

TABLE 1
Exemplary excipients and carriers
of nasal and pulmonary formulations

| Dosage | |
|---|---|
| Form | Ingredients in Addition to a Peptide Product |
| nasal | microcrystalline cellulose, sodium carboxymethylcellulose, |
| spray | dextrose, water, and optionally a pH adjuster (e.g., HCl) |
| nasal | microcrystalline cellulose, carboxymethyl cellulose |
| spray | sodium, dextrose, polysorbate 80, disodium edetate, |
| | potassium sorbate, a pH adjuster (e.g., HCl), water, |
| | and optionally an alcohol (e.g., ethanol) |
| nasal | microcrystalline cellulose, carboxymethyl cellulose |
| spray | sodium, dextrose, polysorbate 80, benzalkonium chloride, |
| | phenylethyl alcohol, water, and optionally an alcohol |
| | (e.g., ethanol) |
| nasal | hypromellose, benzalkonium chloride, NaCl, EDTA, citric |
| spray | acid, sodium phosphate dibasic, water, and optionally |
| | an alcohol (e.g., ethanol) |
| inhalation | mannitol, glycine, sodium citrate and NaOH |
| (DPI) | |
| inhalation | lactose, starch, a starch derivative (e.g., hydroxypropylmethyl |
| (DPI) | cellulose) or polyvinylpyrrolidine, and optionally magnesium |
| | stearate or/and leucine |
| inhalation | a propellant (e.g., 1,1,1,2-tetrafluoroethane), a surfactant |
| (MDI) | (e.g., lecithin or oleic acid), and a co-solvent (e.g., ethanol) |
| inhalation | polysorbate 80, edetate disodium, sodium chloride, pH |
| (nebulizer) | buffering agents (e.g., citric acid/sodium citrate), and water |

In further embodiments, a peptide product is administered via a pulmonary route, such as by oral inhalation or nasal inhalation. Pulmonary administration of a drug can treat a lung disorder or/and a systemic disorder, as the lungs serve as a portal to the systemic circulation. Advantages of pulmonary drug delivery include, for example: 1) avoidance of first-pass metabolism; 2) fast drug action; 3) large surface area of the alveolar region for absorption, high permeability of the lungs (thin air-blood barrier), and profuse vasculature of the airways; and 4) reduced extracellular enzyme levels compared to the GI tract due to the large alveolar surface area. An advantage of oral inhalation over nasal inhalation includes deeper penetration/deposition of the drug into the lungs, although nasal inhalation can deliver the drug into systemic circulation transmucosally in the nasal cavity as well as in the lungs. Oral or nasal inhalation can be achieved by means of, e.g., a metered-dose inhaler (MDI), a nebulizer or a dry powder inhaler (DPI). For example, a peptide product can be formulated for aerosol administration to the respiratory tract by oral or nasal inhalation. The drug is delivered in a small particle size (e.g., between about 0.5 micron and about 5 microns), which can be obtained by micronization, to improve, e.g., drug deposition in the lungs and drug suspension stability. The drug can be provided in a pressurized pack with a suitable propellant, such as a hydrofluoroalkane (HFA, e.g., 1,1,1,2-tetrafluoroethane [HFA-134a]), a chlorofluorocarbon (CFC, e.g., dichlorodifluoromethane, trichlorofluoromethane or dichlorotetrafluoroethane), or a suitable gas (e.g., oxygen, compressed air or carbon dioxide). The drug in the aerosol formulation is dissolved, or more often suspended, in the propellant for delivery to the lungs. The aerosol can contain excipients such as a surfactant (which enhances penetration into the lungs by reducing the high surface tension forces at the air-water interface within the alveoli, may also emulsify, solubilize or/and stabilize the drug, and can be, e.g., a phospholipid such as lecithin) or/and a stabilizer, although the surfactant moiety of the peptide product can perform functions of a surfactant. For example, an MDI formulation can comprise a peptide product, a propellant (e.g., an HFA such as 1,1,1,2-tetrafluoroethane) and a co-solvent (e.g., an alcohol such as ethanol), and optionally a surfactant (e.g., a fatty acid such as oleic acid). The MDI formulation can optionally contain a dissolved gas (e.g., C0₂). After device actuation, the bursting of C0₂ bubbles within the emitted aerosol droplets breaks up the droplets into smaller droplets, thereby increasing the respirable fraction of drug. As another example, a nebulizer formulation can comprise a peptide product, a chelator or preservative (e.g., edetate disodium), an isotonicity agent (e.g., NaCl), pH buffering agents (e.g., citric acid/sodium citrate) and water, and optionally a surfactant (e.g., a Tween® such as polysorbate 80). The drug can be delivered by means of, e.g., a nebulizer or an MDI with or without a spacer, and the drug dose delivered can be controlled by a metering chamber (nebulizer) or a metering valve (MDI).

Table 1 shows exemplary MDI, nebulizer and DPI formulations. Metered-dose inhalers (also called pressurized metered-dose inhalers [pMDI]) are the most widely used inhalation devices. A metering valve delivers a precise amount of aerosol (e.g., about 20-100 pL) each time the device is actuated. MDIs typically generate aerosol faster than the user can inhale, which can result in deposition of much of the aerosol in the mouth and the throat. The problem of poor coordination between device actuation and inhalation can be addressed by using, e.g., a breath-actuated MDI or a coordination device. A breath-actuated MDI (e.g., Easi Breathe®) is activated when the device senses the user's inspiration and discharges a drug dose in response. The inhalation flow rate is coordinated through the actuator and the user has time to actuate the device reliably during inhalation. In a coordination device, a spacer (or valved holding chamber), which is a tube attached to the mouthpiece end of the inhaler, serves as a reservoir or chamber holding the drug that is sprayed by the inhaler and reduces the speed at which the aerosol enters the mouth, thereby allowing for the evaporation of the propellant from larger droplets. The spacer simplifies use of the inhaler and increases the amount of drug deposited in the lungs instead of in the upper airways. The spacer can be made of an anti-static polymer to minimize electrostatic adherence of the emitted drug particles to the inner walls of the spacer. Nebulizers generate aerosol droplets of about 1-5 microns. They do not require user coordination between device actuation and inhalation, which can significantly affect the amount of drug deposited in the lungs. Compared to MDIs and DP Is, nebulizers can deliver larger doses of drug, albeit over a longer administration time. Examples of nebulizers include without limitation human-powered nebulizers, jet nebulizers (e.g., AeroEclipse® II BAN [breath-actuated], CompAIR™NE-C801 [virtual valve], PARI LC® Plus [breath-enhanced] and SideStream Plus [breath-enhanced]), ultrasonic wave nebulizers, and vibrating mesh nebulizers (e.g., Akita2® Apixneb, I-neb AAD System with metering chambers, MicroAir® NE-U22, Omron U22 and PARI eFlow® rapid). As an example, a pulsed ultrasonic nebulizer can aerosolize a fixed amount of the drug per pulse, and can comprise an opto-acoustical trigger that allows the user to synchronize each breath to each pulse. For oral or nasal inhalation using a dry powder inhaler (DPI), a peptide product can be provided in the form of a dry micronized powder, where the drug particles are of a certain small size (e.g., between about 0.5 micron and about 5 microns) to improve, e.g., aerodynamic properties of the dispersed powder and drug deposition in the lungs. Particles between about 0.5 micron and about 5 microns deposit by sedimentation in the terminal bronchioles and the alveolar regions. By contrast, the majority of larger particles (>5 microns) do not follow the stream of air into the many bifureations of the airways, but rather deposit by impaction in the upper airways, including the oropharyngeal region of the throat. A DPI formulation can contain the drug particles alone or be blended with a powder of a suitable larger base/carrier, such as lactose, starch, a starch derivative (e.g., hydroxypropylmethyl cellulose) or polyvinylpyrrolidine. The carrier particles enhance flow, reduce aggregation, improve dose uniformity and aid in dispersion of the drug particles. A DPI formulation can optionally contain an excipient such as magnesium stearate or/and leucine that improves the performance of the formulation by interfering with inter-particle bonding (by anti-adherent action). The powder formulation can be provided in unit dose form, such as a capsule (e.g., a gelatin capsule) or a cartridge in a blister pack, which can be manually loaded or pre-loaded in an inhaler. The drug particles can be drawn into the lungs by placing the mouthpiece or nosepiece of the inhaler into the mouth or nose, taking a sharp, deep inhalation to create turbulent airflow, and holding the breath for a period of time (e.g., about 5-10 seconds) to allow the drug particles to settle down in the bronchioles and the alveolar regions. When the user actuates the DPI and inhales, airflow through the device creates shear and turbulence, inspired air is introduced into the powder bed, and the static powder blend is fluidized and enters the user's airways. There, the drug particles separate from the carrier particles due to turbulence and are carried deep into the lungs, while the larger carrier particles impact on the oropharyngeal surfaces and are cleared. Thus, the user's inspiratory airflow achieves powder de-agglomeration and aeroionisation and determines drug deposition in the lungs. (While a passive DPI requires rapid inspiratory airflow to de agglomerate drug particles, rapid inspiration is not recommended with an MDI or nebulizer, since it creates turbulent airflow and fast velocity which increase drug deposition by impaction in the upper airways.) Compared to an MDI, a DPI (including a breath-activated DPI) may be able to deliver larger doses of drug, and larger-size drugs (e.g., macromolecules), to the lungs.

Lactose (e.g., alpha-lactose monohydrate) is the most commonly used carrier in DPI formulations. Examples of grades/types of lactose monohydrate for DPI formulations include without limitation DCL 11, Flowlac® 100, Inhalac® 230, Lactohale® 300, Lactopress® SD 250 (spray-dried lactose), Respitose® SV003 and Sorbolac® 400. A DPI formulation can contain a single lactose grade or a combination of different lactose grades. For example, a fine lactose grade like Lactohale® 300 or Sorbolac® 400 may not be a suitable DPI carrier and may need to be blended with a coarse lactose grade like DCL 11, Flowlac® 100, Inhalac® 230 or Respitose® SV003 (e.g., about a 1:9 ratio of fine lactose to coarse lactose) to improve flow.

Tables 2 and 3 show non-limiting examples of grades/types of lactose that can be used in DPI formulations. The distribution of the carrier particle sizes affects the fine particle fraction/dose (FPF or FPD) of the drug, with a high FPF being desired for drug delivery to the lungs. FPF/FPD is the respirable fraction/dose mass out of the DPI device with an aerodynamic particle size <5 microns in the inspiration air. High FPF, and hence good DPI performance, can be obtained from, e.g., DPI formulations having an approximately 1:9 ratio of fine lactose (e.g., Lactohalc® 300) to coarse lactose (e.g., Respitose® SV003) and about 20% w/w overages to avoid deposition of the drug in the capsule shell or the DPI device and to deliver essentially all of the drug to the airways.

TABLE 2

| | | Range of Particle Sizes (μm) | | |
|---|---|---|---|---|
| Product | Type | 10% | 50% | 90% |
| Lactohale ® | LH200 | <9 | <69 | <141 |
| InhaLac ® | 230 | <35 | <93 | <138 |
| Respitose ® | ML001 | <4 | <43 | <146 |
| | ML003 | <4 | <35 | <106 |
| | SV003 | <30 | <59 | <90 |
| | SV004 | <32 | <61 | <93 |

TABLE 3

| | | Range of Particle Sizes | | | |
|---|---|---|---|---|---|
| Product | Type | <45 μm | <100 μm | <150 μm | <250 μm |
| Respitose ® | ML003 | 65% | 98% | 100% | NA |
| Respitose ® | ML002 | 65% | 98% | NA | 100% |

Other carriers for DPI formulations include without limitation glucose, mannitol (e.g., crystallized mannitol [Pearlitol 110 C] and spray-dried mannitol [Pearlitol 100 SD]), maltitol (e.g., crystallized maltitol [Maltisorb P90]), sorbitol and xylitol. Most DPIs are breath-activated (“passive”), relying on the user's inhalation for aerosol generation. Examples of passive DPIs include without limitation Airmax®, Novolizer® and Otsuka DPI (compact cake). The air classifier technology (ACT) is an efficient passive powder dispersion mechanism employed in DPIs. In ACT, multiple supply channels generate a tangential airflow that results in a cyclone within the device during inhalation. There are also power-assisted (“active”) DPIs (based on, e.g., pneumatics, impact force or vibration) that use energy to aid, e.g., particle de-agglomeration. For example, the active mechanism of Exubera® inhalers utilizes mechanical energy stored in springs or compressed-air chambers. Examples of active DPIs include without limitation Actispire® (single-unit dose), Aspirair® (multi-dose), Exubera® (single-unit dose), MicroDose® (multi-unit dose and electronically activated), Omnihaler® (single-unit dose), Pfeiffer DPI (single-unit dose), and Spiros® (multi-unit dose). A peptide product can also be administered by other routes, such as orally. An oral formulation can contain a peptide product and conventional excipients known in the art, and optionally an absorption enhancer such as sodium V-[8-(2-hydroxybenzoyl) aminocaprylate] (SNAC). SNAC protects against enzymatic degradation via local buffering action and enhances GI absorption. An oral dosage form (e.g., a tablet, capsule or pill) can optionally have an enteric coating to protect its content from the strong acids and proteolytic enzymes of the stomach. In some embodiments, a peptide product is delivered from a sustained-release composition. As used herein, the term “sustained-release composition” encompasses sustained-release, prolonged-release, extended-release, delayed-release, slow-release and controlled-release compositions, systems and devices. In some embodiments, a sustained-release composition delivers a peptide product over a period of at least about 1 week, 2 weeks, 3 weeks, 1 month, 2 months, 3 months or longer. In some embodiments, a sustained-release composition is formulated as nanoparticles or microparticles composed of a biodegradable polymer and incorporating a peptide product. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. In further embodiments, a sustained-release composition is in the form of a depot that is generated when a mixture of a peptide product and a polymer is injected into a subject intramuscularly or subcutaneously. In certain embodiments, the polymer is or comprises PEG, polylactic acid (PLA) or polyglycolic acid (PGA), or a copolymer thereof (e.g., PLGA or PLA-PEG).

A pharmaceutical composition can be presented in unit dosage form as a single dose wherein all active and inactive ingredients are combined in a suitable system, and components do not need to be mixed to form the composition to be administered. A unit dosage form generally contains a therapeutically effective dose of the drug but can contain an appropriate fraction thereof so that taking multiple unit dosage forms achieves the therapeutically effective dose. Examples of a unit dosage form include a tablet, capsule or pill for oral uptake; a solution in a pre-filled syringe of a single-use pen or a pen with a dose counter for parenteral (e.g., intravenous, subcutaneous or intramuscular) injection; and a capsule, cartridge or blister pre-loaded in or manually loaded into an inhaler. Alternatively, a pharmaceutical composition can be presented as a kit in which the active ingredient, excipients and carriers (e.g., solvents) are provided in two or more separate containers (e.g., ampules, vials, tubes, bottles or syringes) and need to be combined to form the composition to be administered. The kit can contain instructions for storing, preparing and administering the composition (e.g., a solution to be injected parenterally). A kit can contain all active and inactive ingredients in unit dosage form or the active ingredient and inactive ingredients in two or more separate containers, and can contain instructions for administering or using the pharmaceutical composition to treat a medical condition disclosed herein. A kit can further contain a device for delivering the composition, such as an injection pen or an inhaler. In some embodiments, a kit contains a peptide product or a pharmaceutically acceptable salt thereof, or a pharmaceutical composition comprising the same, and instructions for administering or using the peptide product or the composition to treat a medical condition disclosed herein, such as insulin resistance, diabetes, metabolic syndrome, cardiovascular disease, obesity (including “chronic obesity” meaning obesity lasting more than one year or resulting in an obesity-related condition such as but not limited to insulin resistance, diabetes, metabolic syndrome, and/or cardiovascular disease), or a condition associated therewith (e.g.,). In certain embodiments, the kit further contains a device for delivering the peptide product or the composition, such as an injection pen or an inhaler.

The disclosure further provides uses of the dual agonist peptide products described herein to prevent and/or treat conditions associated with GLP1R and/or GCGR, such as but not limited to insulin resistance, diabetes, obesity, metabolic syndrome and cardiovascular diseases, and conditions associated therewith, such as NASH and PCOS. In some embodiments, the dual agonist peptide products can be used to treat hyperglycemia, insulin resistance, hyperinsulinemia, prediabetes, diabetes (including types 1 and 2, gestational and juvenile diabetes), diabetic complications, diabetic neuropathy, diabetic nephropathy, diabetic retinopathy, hyperlipidemia, hypercholesterolemia, hypertriglyceridemia, elevated blood levels of free fatty acids, obesity, metabolic syndrome, syndrome X, cardiovascular diseases (including coronary artery disease), atherosclerosis, acute cardiovascular syndrome, ischemia (including myocardial ischemia and cerebral ischemia/stroke), ischemia-reperfusion injury (including myocardial and cerebral IRI), infarction (including myocardial and cerebral infarction), angina, heart failure (e.g., congestive heart failure), peripheral vascular disease, thrombosis (e.g., deep vein thrombosis), embolism (e.g., pulmonary embolism), systemic inflammation (e.g., one characterized by elevated C-reactive protein blood level), and hypertension. The dual agonist peptide products can achieve their therapeutic effects through various mechanisms, including stimulation of blood glucose-dependent insulin secretion, increase in insulin sensitivity, stimulation of fat burning and reduction of body weight. The dual agonist peptide products can also promote, e.g., pancreatic beta-cell protection, cardioprotection and wound healing.

The peptide products described herein can be used to treat other conditions associated with insulin resistance or/and obesity. Other conditions associated with insulin resistance or/and obesity include without limitation arthritis (e.g., osteoarthritis), low back pain, breathing disorders (e.g., asthma, obesity hypoventilation syndrome [Pickwickian syndrome] and obstructive sleep apnea), dermatological disorders (e.g., diabetic ulcers, acanthosis nigricans, cellulitis, hirsutism, intertrigo and lymphedema), gastroenterological disorders (e.g., cholelithiasis [gallstone], gastroesophageal reflux disease [GERD] and gastroparesis), gout, hypercortisolism (e.g., Cushing's syndrome), kidney disorders (e.g., chronic kidney disease), liver disorders (e.g., fatty liver disease [FLD] including alcoholic and non-alcoholic FLD), neurological disorders (e.g., carpal tunnel syndrome, dementias [e.g., Alzheimer's disease and vascular dementia], meralgia paresthetica, migraines and multiple sclerosis), urological disorders (e.g., erectile dysfunction, hypogonadism and urinary incontinence), polycystic ovary syndrome, infertility, menstrual disorders, mood disorders (e.g., depression), and cancers (e.g., cancers of the endometrium, esophagus, colorectum, gallbladder, kidney, liver [e.g., hepatocellular carcinoma], pancreas and skin [e.g., melanoma], and leukemia). In certain embodiments, a dual agonist peptide product described herein is used to treat polycystic ovary syndrome (PCOS). In other embodiments, a peptide product is used to treat chronic kidney disease (CKD), also known as chronic kidney/renal failure (CKF/CRF). The most common causes of CKD are diabetes and long-term, uncontrolled hypertension. In further embodiments, a dual agonist peptide product described herein is used to treat fatty liver disease (FLD). In some embodiments, the FLD is non-alcoholic fatty liver disease (NAFLD), also understood to include metabolic fatty liver disease (MFLD). In certain embodiments, the NAFLD is non-alcoholic steatohepatitis (NASH). FLD, also known as hepatic steatosis, is characterized by excessive fat accumulation in the liver. FLD includes alcoholic fatty liver disease (AFLD) and NAFLD. Chronic alcoholism causes fatty liver due to production of toxic metabolites such as aldehydes during metabolism of alcohol in the liver. NAFLD is described below. FLD is associated with diabetes, obesity and metabolic syndrome. Fatty liver can develop into cirrhosis or a liver cancer (e.g., hepatocellular carcinoma [HCC]). Less than about 10% of people with cirrhotic AFLD develop HCC, but up to about 45% of people with NASH without cirrhosis may develop HCC. HCC is the most common type of primary liver cancer in adults and occurs in the setting of chronic liver inflammation. NAFLD is characterized by fatty liver that occurs when fat, in particular free fatty acids and triglycerides, accumulates in liver cells (hepatic steatosis) due to causes other than excessive alcohol consumption, such as nutrient overload, high caloric intake and metabolic dysfunction (e.g., dyslipidemia and impaired glucose control). A liver can remain fatty without disturbing liver function, but a fatty liver can progress to become NASH, a condition in which steatosis is accompanied by inflammation, hepatocyte ballooning and cell injury with or without fibrosis of the liver. Fibrosis is the strongest predietor of mortality from NASH. NAFLD can be characterized by steatosis alone; steatosis with lobular or portal inflammation but without ballooning; steatosis with ballooning but without inflammation; or steatosis with inflammation and ballooning. NASH is the most extreme form of NAFLD. NASH is a progressive disease, with about 20% of patients developing cirrhosis of the liver and about 10% dying from a liver disease, such as cirrhosis or a liver cancer (e.g., HCC). NAFLD is the most common liver disorder in developed countries, and NASH is projected to supplant hepatitis C as the major cause of liver transplant in the U.S. by 2020. About 12-25% of people in the U.S. have NAFLD, with NASH affecting about 2-5% of people in the U.S. NAFLD, including NASH, is associated with insulin resistance, obesity and metabolic syndrome. For instance, insulin resistance contributes to progression of fatty liver to hepatic inflammation and fibrosis and thus NASH. Furthermore, obesity drives and exacerbates NASH, and weight loss can alleviate NASH. Therefore, the peptide products described herein, including GLP-1 receptor (GLP1R) agonists, glucagon receptor (GCGR) agonists and dual GLP1R/GCGR agonists, can be used to treat NAFLD, including NASH. In some embodiments, the dual agonist peptide products used to treat a condition associated with insulin resistance or/and obesity disclosed herein, such as fatty liver disease including NAFLD and NASH, is pemvidutide, and/or derivatives thereof, and pharmaceutically acceptable salts thereof.

In some embodiments, the present dual agonist peptide(s) can be used to control blood glucose with reduction of one or more adverse events (i.e., an unexpected event that negatively impacts patient and/or animal welfare). Exemplary, non-limiting adverse events can include nausea, vomiting, diarrhea, abdominal pain and/or constipation. Adverse events may also include any known to those of ordinary skill in the art, such as those listed in industry resources and/or otherwise known to those of ordinary skill in the art (see, e.g., Medical Dictionary for Regulatory Activities (MedDRA) (Pharm., Med. Transl. Med. 2018) and/or Clark, M. J. Biomed. Inf., 54. April 2015, pp. 167-173). Such adverse events can be determined in humans using standard techniques as are typically used in clinical trials (e.g., doctor visit, surveys/questionnaires). As compared to the frequency and/or severity of such an adverse event that occurs upon administration of an agonist with unbalanced affinity for GLP-1R and GCGR (e.g., semaglutide) to a subject, the dual agonist peptides of this disclosure (e.g., any of SEQ ID NO. 1, or derivatives thereof) can decrease such frequency and/or severity thereof by, e.g., 5%, 10%, 20%, 40%, 50%, 60%, 70%, 80%, 90% of higher (up to 100%). In some embodiments, the dual agonist peptides of this disclosure (e.g., pemvidutide) do not cause any adverse events.

A present dual agonist peptide product can be administered by any suitable route for treatment of a condition disclosed herein. Potential routes of administration of a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by cyc drop), pulmonary (e.g., by oral or nasal inhalation), buccal, sublingual, rectal (e.g., by suppository), and vaginal (e.g., by suppository)). In some embodiments, a peptide product is administered parenterally, such as subcutaneously, intravenously or intramuscularly. In other embodiments, a peptide product is administered by oral inhalation or nasal inhalation or insufflation. The therapeutically effective amount and the frequency of administration of, and the length of treatment with, a peptide product to treat a condition disclosed herein may depend on various factors, including the nature and severity of the condition, the potency of the compound, the route of administration, the age, body weight, general health, gender and diet of the subject, and the response of the subject to the treatment, and can be determined by the treating physician. In some embodiments, a peptide product is administered parenterally (e.g., subcutaneously (sc), intravenously (iv) or intramuscularly (im)) in a dose from about 0.01 mg to about 0.1, 1, 5 or 10 mg, or about 0.1-1 mg or 1-10 mg, over a period of about one week for treatment of a condition disclosed herein (e.g., one associated with insulin resistance or/and obesity, such as NASH or NAFLD). In further embodiments, a peptide product is administered parenterally (e.g., sc, iv or im) in a dose of about 0.1-0.5 mg, 0.5-1 mg, 1-5 mg or 5-10 mg over a period of about one week. In certain embodiments, a peptide product is administered parenterally (e.g., subcutaneously (SC), intravenous (IV) or intramuscular (IM)) in a dose of about 0.1-1 mg, or about 0.1-0.5 mg or 0.5-1 mg, over a period of about one week. One of skill in the art understands that an effective dose in a mouse, or other pre-clinical animal model, may be scaled for a human. In that way, through allometric scaling (also referred to as biological scaling) a dose in a larger animal may be extrapolated from a dose in a mouse to obtain an equivalent dose based on body weight or body surface area of the animal.

A peptide product can be administered in any suitable frequency for prevention and/or treatment of a CV disease-related condition disclosed herein (e.g., high cholesterol, triglycerides, LDL and/or VLDL particle concentration and/or diameter, and/or other related parameters (e.g., serum phosphatidylethanolamines, phosphatidylcholines, lysophosphatidylethanolamines, sphingolipids and/or lysophosphatidylcholines); e.g., heart failure with preserved ejection fraction (HFpEF)). In some embodiments, a dual agonist peptide product is administered, e.g., sc or iv once a day, once every two days, once every three days, twice a week, once a week or once every two weeks. In certain embodiments, a peptide product is administered, e.g., SC, IV, or IM once a week. A dual agonist peptide product can be administered at any time of day convenient to the patient. A dual agonist peptide product can be taken substantially with food (e.g., with a meal or within about 1 hour or 30 minutes before or after a meal) or substantially without food (e.g., at least about 1 or 2 hours before or after a meal). The length of treatment of a medical condition with a dual agonist peptide product can be based on, e.g., the nature and severity of the condition and the response of the subject to the treatment and can be determined by the treating physician. In some embodiments, a dual agonist peptide product is administered chronically to treat a condition disclosed herein, such as at least about 2 months, 3 months, 6 months, 1 year, 1.5 years, 2 years, 3 years, 5 years, 10 years or longer. A dual agonist peptide product can also be taken pro re nata (as needed) until clinical manifestations of the condition disappear or clinical targets are achieved, such as blood glucose level, blood pressure, blood levels of lipids, body weight or body mass index, waist-to-hip ratio or percent body fat, or any combination thereof. If clinical manifestations of the condition re-appear or the clinical targets are not maintained, administration of the dual agonist peptide product can resume. The disclosure provides a method of treating a medical condition described herein, comprising administering to a subject in need of treatment a therapeutically effective amount of a peptide product described herein or a pharmaceutically acceptable salt thereof, or a pharmaceutical composition comprising the same. The disclosure further provides a peptide product described herein or a pharmaceutically acceptable salt thereof, or a composition comprising the same, for use as a medicament. In addition, the disclosure provides for the use of a peptide product described herein or a pharmaceutically acceptable salt thereof in the preparation of a medicament. The medicament containing the peptide product can be used to treat any medical condition described herein. The peptide product can optionally be used in combination with one or more additional therapeutic agents.

A dual agonist peptide product described herein can be administered as the sole active agent, or optionally be used in combination with one or more other dual agonist peptide products, and/or additional therapeutic agents to treat any disorder disclosed herein, such as insulin resistance, diabetes, obesity, metabolic syndrome or a CV disease (e.g., heart failure with preserved ejection fraction (HFpEF)), or any condition associated therewith, such as high cholesterol, triglycerides, LDL and/or VLDL particle concentration and/or diameter, and/or other related parameters (e.g., scrum phosphatidylethanolamines, phosphatidylcholines, lysophosphatidylethanolamines, sphingolipids and/or lysophosphatidylcholines). In some embodiments, the one or more additional therapeutic agents are selected from antidiabetic agents, anti-obesity agents (including lipid-lowering agents and pro-satiety agents), anti-atherosclerotic agents, anti-inflammatory agents, antioxidants, antifibrotic agents, anti-hypertensive agents, and combinations thereof. Antidiabetic agents include without limitation: AMP-activated protein kinase (AMPK) agonists, including biguanides (e g., buformin and metformin); peroxisome proliferator-activated receptor gamma (PPAR-Y) agonists, including thiazolidinediones (e.g., balaglitazone, ciglitazone, darglitazone, englitazone, lobeglitazone, netoglitazone, pioglitazone, rivoglitazone, rosiglitazone and troglitazone), MSDC-0602K and saroglitazar (dual PPAR-α/γ agonist); glucagon-like peptide-1 (GLP-1) receptor agonists, including exendin-4, albiglutide, dulaglutide, exenatide, liraglutide, lixisenatide, semaglutide, taspoglutide, CNT0736, CNT03649, HM11260C (LAPS-Exendin), NN9926 (OG9S7GT), TT401 and ZYOGI; dipeptidyl peptidase 4 (DPP-4) inhibitors, including alogliptin, anagliptin, dutogliptin, evogliptin, gemigliptin, gosogliptin, linagliptin, omarigliptin, saxagliptin, septagliptin, sitagliptin, teneligliptin, trelagliptin and vildagliptin; sodium-glucose transport protein 2 (SGLT2) inhibitors, including canagliflozin (also inhibits SGLT1), dapagliflozin, empagliflozin, ertugliflozin, ipragliflozin, luseogliflozin, remogliflozin etabonate, sotagliflozin (also inhibits SGLT1) and tofogliflozin; blockers of ATP-dependent K⁺ (KA_{TP}) channels on pancreatic beta cells, including rneglitinides (e.g., mitiglinide, nateglinide and repagiinide) and sulfonylureas {including first generation (e.g., acetohexamide, carbutamide, chlorpropamide, giycyclamide [tolhexamide], metahexamide, tolazamide and tolbutamide) and second generation (e.g., glibenclamide, glyburide, glibornuride, gliclazide, glimepiride, glipizide, gliquidone, glisoxepide and glyclopyramide); insulin and analogs thereof, including fast-acting insulin (e.g., insulin aspari insulin glulisine and insulin lispro), intermediate-acting insulin (e.g., NPH insulin), and long-acting insulin (e.g., insulin degludec, insulin detemir and insulin glargine); and/or, analogs, derivatives and salts thereof. In certain embodiments, the antidiabetic agent is or includes a biguanide (e.g., metformin), a thiazolidinedione (e.g., pioglitazone or rosiglitazone) or a SGLT2 inhibitor (e.g., empagliflozin or tofogliflozin), or any combination thereof. Anti-obesity agents include, but are not limited to: appetite suppressants (anorectics), including amphetamine, dexamphetamine, amfepramone, clobenzorex, mazindol, phentermine (with or without topiramate) and lorcaserin; pro-satiety agents, including ciliary neurotrophic factor (e.g., axokine) and longer-acting analogs of amylin, calcitonin, cholecystokinin (CCK), GLP-1, leptin, oxyntomodulin, pancreatic polypeptide (PP), peptide YY (PYY) and neuropeptide Y (NPY); lipase inhibitors, including caulerpenyne, cetilistat, ebelactone A and B, estcrastin, lipstatin, orlistat, percyquinin, panclicin A-E, valilactone and vibralactone; antihyperlipidemic agents; and analogs, derivatives and salts thereof. Antihyperlipidemic agents include without limitation: HMG-COA reductase inhibitors, including statins {e.g., atorvastatin, cerivastatin, fluvastatin, mevastatin, monacolins (e.g., monacolin K (lovastatin), pitavastatin, pravastatin, rosuvastatin and simvastatin} and flavanones (e.g., naringenin); squalene synthase inhibitors, including lapaquistat, zaragozic acid and RPR-107393; acetyl-CoA carboxylase (ACC) inhibitors, including anthocyanins, avenaciolides, chloroacetylated biotin, cyclodim, diclofop, haloxyfop, soraphens (e.g., soraphen Aₗₐ), 5-(tetradecyloxy)-2-furancarboxylic acid (TOFA), CP-640186, GS-0976, NDI-010976; 7-(4-propyloxy-phenylethynyl)-3,3-dimethyl-3,4dihydro-2H-benzo[b][1,4]dioxepine; N-ethyl-N′-(3-{[4-(3,3-dimethyl-1-oxo-2-oxa-7-azaspiro[4.5]dec-7-yl)piperidin-1-yl]-carbonyl}-1-benzothien-2-yl)urea; 5-(3-acetamidobut-1-ynyl)-2-(4-propyloxyphenoxy)thiazole; and 1-(3-{[4-(3,3-dimethyl-1-oxo-2-oxa-7-azaspiro[4.5]dec-7-yl)piperidin-1-yl]-carbonyl}-5-(pyridin-2-yl)-2-thienyl)-3-ethylurea; PPAR-α agonists, including fibrates (e.g., bezafibrate, ciprofibrate, clinofibrate, clofibric acid, clofibrate, aluminum clofibrate [alfibrate], clofibride, etofibrate, fenofibric acid, fenofibrate, gemfibrozil, ronifibrate and simfibrate), isoflavones (e.g., daidzein and genistein), and perfluoroalkanoic acids (e.g., perfluorooctanoic acid and perfluorononanoic acid); PPAR-δ agonists, including elafibranor (dual PPAR-α/γ agonist), GFT505 (dual PPAR-α/γ agonist), GW0742, GW501516 (dual PPAR-β/δ agonist), sodelglitazar (GW677954), MBX-8025, and isoflavones (e.g., daidzein and genistein); PPAR-γ agonists, including thiazolidinediones {supra), saroglitazar (dual PPAR-α/γ agonist), 4-oxo-2-thioxothiazolines (e.g., rhodanine), berberine, honokiol, perfluorononanoic acid, cyclopentenone prostaglandins (e.g., cyclopentenone 15-deoxy-A-prostaglandin J₂[15d-PGJ₂]), and isoflavones (e.g., daidzein and genistein); liver X receptor (LXR) agonists, including endogenous ligands (e.g., oxysterols such as 22(i?)-hydroxycholesterol, 24(A)-hydroxy cholesterol, 27-hydroxycholesterol and cholestenoic acid) and synthetic agonists (e.g., acetyl-podocarpic dimer, hypocholamide, A(X-di methyl-3b-hydroxy-cholenamide [DMHCA], GW3965 and T0901317); retinoid X receptor (RXR) agonists, including endogenous ligands (e.g., 9-cis-retinoic acid) and synthetic agonists (e.g., bexarotene, AGN 191659, AGN 191701, AGN 192849, BMS649, LG100268, LG100754 and LGD346); inhibitors of acyl-CoA cholesterol acyltransferase (ACAT, aka sterol G-acyl transferase [SOAT], including ACAT1 [SOAT1] and ACAT2 [SOAT2]), including avasimibe, pactimibe, pellitorine, terpendole C and flavanones (e.g., naringenin); inhibitors of stearoyl-CoA desaturase-1 (SCD-1, aka stearoyl-CoA delta-9 desaturase) activity or expression, including aramchol, CAY-10566, CVT-11127, SAR-224, SAR-707, XEN-103; 3-(2-hydroxyethoxy)-4-methoxy-N-[5-(3-trifluoromethylbenzyl)thiazol-2-yl]benzamide and 4-ethylamino-3-(2-hydroxyethoxy)-N-[5-(3-trifluoromethylbenzyl)thiazol-2-yl]benzamide; 1′-{6-[5-(pyridin-3-ylmethyl)-1,3,4-oxadiazol-2-yl]pyridazin-3-yl}-5-(trifluoromethyl)-3,4-dihydrospiro[chromene-2,4′-piperidine]; 5-fluoro-1′-{6-[5-(pyridin-3-ylmethyl)-1,3,4-oxadiazol-2-yl]pyridazin-3-yl}-3,4-dihydrospiro[chromene-2,4′-piperidine]; 6-[5-(cyclopropylmethyl)-4,5-dihydro-1′H,3H-spiro[1,5-benzoxazepine-2,4′-piperidin]-1′-yl]-N-(2-hydroxy-2-pyridin-3-ylethyl)pyridazine-3-carboxamide; 6-[4-(2-methylbenzoyl)piperidin-1-yl]pyridazine-3-carboxylic acid (2-hydroxy-2-pyridin-3-ylethyl)amide; 4-(2-chlorophenoxy)-N-[3-(methyl carbamoyl)phenyl]piperidine-1-carboxamide; the cis-9, trans-11 isomer and the trans-10,cis-12 isomer of conjugated linoleic acid, substituted heteroaromatic compounds disclosed in WO 2009/129625 A1, anti-sense polynucleotides and peptide-nucleic acids (PNAs) that target mRNA for SCD-1, and SCD-1-targeting siRNAs; cholesterylester transfer protein (CETP) inhibitors, including anacetrapib, dalcetrapib, cvacetrapib, torcetrapib and AMG 899 (TA-8995); inhibitors of microsomal triglyceride transfer protein (MTTP) activity or expression, including implitapide, lomitapide, dirlotapide, mitratapide, CP-346086, JTT-130, SLx-4090, anti-sense polynucleotides and PNAs that target mRNA for MTTP, MTTP-targeting microRNAs (e.g., miRNA-30c), and MTTP-targeting siRNAs; GLP-1 receptor agonists; fibroblast growth factor 21 (FGF21) and analogs and derivatives thereof, including BMS-986036 (pegylated FGF21); inhibitors of pro-protein convertase subtilisin/kexin type 9 (PCSK9) activity or expression, including berberine (reduces PC8K9 level), annexin A2 (inhibits PCSK9 activity), anti-PCSK9 antibodies (e.g., alirocumab, bococizumab, cvolocumab, LGT-209, LY3015014 and RG7652), peptides that mimic the epidermal growth factor-A (EGF-A) domain of the LDL receptor which binds to PCSK9, PCSK9-binding adnectins (e.g., BMS-962476), anti-sense polynucleotides and PNAs that target mRNA for PCSK9, and PCSK9-targeting siRNAs (e.g, inclisiran [ALN-PCS] and ALN-PCS02); apolipoprotein mimetic peptides, including apoA-I mimetics (e.g., 2F, 3F, 3F-1, 3F-2, 3F-14, 4F, 4F-P-4F, 4F-IHS-4F, 4F2, 5F, 6F, 7F, 18F, 5A, 5A-C1, 5A-CH1, 5A-CH2, 5A-H1, 18 A, 37 pA [18A-P-18A], ELK, ELK-1A, ELK-1F, ELK-1K1A1E, ELK-1L1K, ELK-1W, ELK-2A, ELK-2A2K2E, ELK-2E2K, ELK-2F, ELK-3 E3EK, ELK-3E3K3A, ELK-3E3LK, ELK-PA, ELK-P2A, ELKA, ELKA-CH2, ATI-5261, CS-6253, ETC-642, FAMP, FREL and KRES and apoE mimetics (e.g., Ac-hE18A-NH₂, AEM-28, Ac-[R]hE1 8 A-NH₂, AEM-28-14, EpK, hEp, mR18L, COG-112, COG-133 and COG-1410); omega-3 fatty acids, including docosahexaenoic acid (DHA), docosapentaenoic acid (DPA), eicosapentaenoic acid (EPA), a-linolenic acid (ALA), fish oils (which contain, e.g., DHA and EPA), and esters (e.g., glyceryl and ethyl esters) thereof; and analogs, derivatives and salts thereof. In certain embodiments, the anti-obesity agent is or includes a lipase inhibitor (e.g., orlistat) or/and an antihyperlipidemic agent (e.g., a statin such as atorvastatin, or/and a fibrate such as fenofibrate). Antihypertensive agents include without limitation: antagonists of the renin-angiotensin-aldosterone system (RAAS), including renin inhibitors (e.g., aliskiren), angiotensin-converting enzyme (ACE) inhibitors (e.g., benazepril, captopril, enalapril, fosinopril, lisinopril, moexipril, perindopril, quinapril, ramipril and trandolapril), angiotensin II receptor type 1 (ATII1) antagonists (e.g., azilsartan, candesartan, eprosartan, fimasartan, irbesartan, losartan, olmesartan medoxomil, olmesartan, telmisartan and valsartan), and aldosterone receptor antagonists (e.g., eplerenone and spironolactone); diuretics, including loop diuretics (e.g., bumetanide, ethacrynic acid, furosemide and torsemide), thiazide diuretics (e.g., bendroflumethiazide, chlorothiazide, hydrochlorothiazide, epitizide, methyclothi azide and polythiazide), thiazide-like diuretics (e.g., chlorthalidone, indapamide and metolazone), cicletanine (an early distal tubular diuretic), potassium-sparing diuretics (e.g., amiloride, eplerenone, spironolactone and triamterene), and theobromine; calcium channel blockers, including dihydropyridines (e.g., amlodipine, levamlodipine, cilnidipine, clevidipine, felodipine, isradipine, lercanidipine, nicardipine, nifedipine, nimodipine, nisoldipine and nitrendipine) and non-dihydropyri dines (e.g., diltiazem and verapamil); α₂-adrenoreceptor agonists, including clonidine, guanabenz, guanfacine, methyldopa and moxonidine; α₁-adrenoreceptor antagonists (alpha blockers), including doxazosin, indoramin, nicergoline, phenoxybenzamine, phentolamine, prazosin, terazosin and tolazoline; β-adrenoreceptor (β1 or/and β₂) antagonists (beta blockers), including atenolol, betaxolol, bisoprolol, carteolol, carvedilol, labetalol, metoprolol, nadolol, nebivolol, oxprenolol, penbutolol, pindolol, propranolol and timolol; mixed alpha/beta blockers, including bucindolol, carvedilol and labetalol; endothelin receptor antagonists, including selective ETA receptor antagonists (e.g., ambrisentan, atrasentan, edonentan, sitaxentan, zibotentan and BQ-123) and dual ET_{A}/ET_{B} antagonists (e.g., bosentan, macitentan and tezosentan); other vasodilators, including hydralazine, minoxidil, theobromine, sodium nitroprusside, organic nitrates (e.g., isosorbide mononitrate, isosorbide dinitrate and nitroglycerin, which are converted to nitric oxide in the body), endothelial nitric oxide synthase (eNOS) stimulators (e.g., cicletanine), activators of soluble guanylate cyclase (e.g., cinaciguat and riociguat), phosphodiesterase type 5 (PDE5) inhibitors (e.g., avanafil, benzamidenafil, dasantafil, dynafil, lodenafil, mirodenafil, sildenafil, tadalafil, udenafil, vardenafil, dipyridamole, papaverine, propentofylline, zaprinast and T-1032), prostaglandin Ei (alprostadil) and analogs thereof (e.g., limaprost amd misoprostol), prostacyclin and analogs thereof (e.g., ataprost, beraprost [e.g., esuberaprost], 5,6,7-trinor-4,8-inter-w-phenylene-9-fluoro-PGl₂, carbacyclin, isocarbacyclin, clinprost, ciprostene, eptaloprost, cicaprost, iloprost, pimilprost, SM-10906 (des-methyl pimilprost), naxaprostene, taprostene, treprostinil, CS-570, OP-2507 and TY-11223), non prostanoid prostacyclin receptor agonists (e.g., 1-phthalazinol, ralinepag, selexipag, ACT-333679 [MRE-269, active metabolite of selexipag], and TRA-418), phospholipase C (PLC) inhibitors, and protein kinase C (PKC) inhibitors (e.g., BIM-1, BIM-2, BIM-3, BIM-8, chelerythrine, cicletanine, gossypol, miyabenol C, myricitrin, ruboxistaurin and verbascoside; minerals, including magnesium and magnesium sulfate; and analogs, derivatives and salts thereof. In certain embodiments, the antihypertensive agent is or includes a thiazide or thiazide like diuretic (e.g., hydrochlorothiazide or chlorthalidone), a calcium channel blocker (e.g., amlodipine or nifedipine), an ACE inhibitor (e.g., benazepril, captopril or perindopril) or an angiotensin II receptor antagonist (e.g., olmesartan medoxomil, olmesartan, telmisartan or valsartan), or any combination thereof. In some embodiments, a peptide product described herein is used in combination with one or more additional therapeutic agents to prevent and/or treat CV disease-related conditions, such as high cholesterol, triglycerides, LDL and/or VLDL particle concentration and/or diameter, and/or other related parameters (e.g., serum phosphatidylethanolamines, phosphatidylcholines, lysophosphatidylethanolamines, sphingolipids and/or lysophosphatidylcholines). In some embodiments, the one or more additional therapeutic agents are selected from antidiabetic agents, anti-obesity agents, anti-inflammatory agents, antifibrotic agents, antioxidants, anti-hypertensive agents, and combinations thereof. Therapeutic agents that can be used to treat NAFLD (e.g., NASH) include without limitation: PPAR agonists, including PPAR-δ agonists (e.g., MBX-8025, elafibranor [dual PPAR-α/δ agonist] and GW501516 [dual PPAR-β/δ agonist]) and PPAR-γ agonists (e.g., thiazolidinediones such as pioglitazone, and saroglitazar [dual PPAR-α/γ agonist])-PPAR-δ and -γ agonism increases insulin sensitivity, PPAR-α agonism reduces liver steatosis and PPAR-δ agonism inhibits activation of macrophages and Kupffer cells; farnesoid X receptor (FXR) agonists, such as obeticholic acid and nonsteroidal FXR agonists like GS-9674 reduce liver gluconcogenesis, lipogenesis, steatosis and fibrosis; fibroblast growth factor 19 (FGF19) and analogs and derivatives thereof, such as NGM-282-FGF19 analogs reduce liver gluconcogenesis and steatosis; fibroblast growth factor 21 (FGF21) and analogs and derivatives thereof, such as BMS-986036 (pegylated FGF21)—FGF21 analogs reduce liver steatosis, cell injury and fibrosis; HMG-COA reductase inhibitors, including statins (e.g., rosuvastatin)—statins reduce steatohepatitis and fibrosis; ACC inhibitors, such as NDI-010976 (liver-targeted) and GS-0976-ACC inhibitors reduce de novo lipogenesis and liver steatosis; SCD-1 inhibitors, such as aramchol—SCD-1 inhibitors reduce liver steatosis and increase insulin sensitivity; SGLT2 inhibitors, such as canagliflozin, ipragliflozin and luscogliflozin—SGLT2 inhibitors reduce body weight, liver ALT level and fibrosis; antagonists of CCR2 or/and CCR5, such as cenicriviroc—antagonists of CCR2 (binds to CCL2 [MCP1]) and CCR5 (binds to CCL5 [RANTES]) inhibit activation and migration of inflammatory cells (e.g., macrophages) to the liver and reduce liver fibrosis; apoptosis inhibitors, including apoptosis signal-regulating kinase 1 (ASK1) inhibitors (e.g., selonsertib) and caspase inhibitors (e.g., emricasan [pan-caspase inhibitor])—apoptosis inhibitors reduce liver steatosis and fibrosis; lysyl oxidase-like 2 (LOXL2) inhibitors, such as simtuzumab—LOXL2 is a key matrix enzyme in collagen formation and is highly expressed in the liver; galectin-3 inhibitors, such as GR-MD-02 and TD139—galectin-3 is critical for development of liver fibrosis; antioxidants, including vitamin E (e.g., a-tocopherol) and scavengers of reactive oxygen species (ROS) and free radicals (e.g., cysteamine, glutathione, melatonin and pentoxifylline [also anti-inflammatory via inhibition of TNF-a and phosphodiesterases])—vitamin E reduces liver steatosis, hepatocyte ballooning and lobular inflammation; and, analogs, derivatives and salts thereof. In some embodiments, a peptide product described herein is used in conjunction with a PPAR agonist (e.g., a PPAR-δ agonist such as elafibranor or/and a PPAR-γ agonist such as pioglitazone), a HMG-COA reductase inhibitor (e.g., a statin such as rosuvastatin), an FXR agonist (e.g., obeticholic acid) or an antioxidant (e.g., vitamin E), or any combination thereof, to treat NAFLD (e.g., NASH). In certain embodiments, the one or more additional therapeutic agents for treatment of NAFLD (e.g., NASH) are or include vitamin E or/and pioglitazone. Other combinations may also be used as would be understood by those of ordinary skill in the art.

Pharmacokinetic (“PK”) parameters can be estimated using Phoenix® WinNonlin® version 8.1 or higher (Certara USA, Inc., Princeton, New Jersey). A non-compartmental approach consistent with the extravascular route of administration can be used for parameter estimation. The individual plasma concentration-time data can be used for pharmacokinetic calculations. In addition to parameter estimates for individual animals, descriptive statistics (e.g. mean, standard deviation, coefficient of variation, median, min, max) can be determined, as appropriate. Concentration values that are below the limit of quantitation can be treated as zero for determination of descriptive statistics and pharmacokinetic analysis. Embedded concentration values that are below the limit of quantitation can be excluded from pharmacokinetic analysis. All parameters can be generated from individual dual agonist peptide (or derivatives and/or metabolites thereof) concentrations in plasma from test article-treated groups on the day of dosing (Day 1). Parameters can be estimated using nominal dose levels, unless out of specification dose formulation analysis results are obtained, in which case actual dose levels can be used. Parameters can be estimated using nominal sampling times; if bioanalytical sample collection deviations are documented, actual sampling times can be used at the affected time points. Bioanalytical data can be used as received for the pharmacokinetic analysis and can be presented in tables and figures in the units provided. Pharmacokinetic parameters can be calculated and presented in the units provided by the analytical laboratory (the order of magnitude can be adjusted appropriately for presentation in the report, e.g. h*ng/mL converted to h*μg/mL). Descriptive statistics (e.g., mean, standard deviation, coefficient of variation, median, min, max) and pharmacokinetic parameters can be determined to three significant figures, as appropriate. Additional data handling items can be documented as needed. PK parameters to be determined, as data permit, can include but are not limited to the following: Cₘₐₓ: Maximum observed concentration; DN Cₘₐₓ: dose normalized maximum concentration, calculated as Cₘₐₓ/dose; Tₘₐₓ: time of maximum observed concentration; AUC₀₋ₜ: area under the curve from time 0 to the time of the last measurable concentration, calculated using the linear trapezoidal rule; AUC₀₋₉₆: area under the curve from time 0 to hour 96, calculated using the linear trapezoidal rule; DN AUC₀₋₉₆: dose normalized AUC₀₋₉₆, calculated as AUC₀₋₉₆/dose; AUC_{0-inf}: area under the curve from time 0 to infinity (Day 1 only), calculated as AUC_{0-inf}=AUC₀₋ₜ+Cₜ/λ_{z}, where Cₜ is the last observed quantifiable concentration and λ_{z} is the elimination rate constant; t_{1/2}: elimination half-life, calculated as ln(2)/λ_{z}. Additional parameters and comparisons (e.g., sex ratios, dose proportionality ratios, etc.) can also be determined, as would be understood by those of ordinary skill in the art.

In some embodiments, this disclosure provides pharmaceutical dosage formulation(s) comprising pemvidutide wherein the peptide product is modified with a hydrophobic surfactant; the dosage is configured to induce weight loss, with reduction of one or more adverse events wherein the subject is at risk for and/or has a CV disease-related conditions (and/or CV disease) and may also be overweight, obese, and/or suffer from type 2 diabetes, wherein the adverse events being selected from nausea, vomiting, diarrhea, abdominal pain and constipation, upon administration to a mammal. In certain embodiments, the formulation comprises 1.8 mg of pemvidutide as a therapeutic dose.

“Reducing,” or “reduction of” adverse effects or events refers to a reduction in the degree, duration, and/or frequency of adverse effects experienced by a subject and incidence in a group of subjects following administration of an agonist with about balanced affinity to GLP1R and GCGR. Such reduction encompasses the prevention of some adverse effects that a subject would otherwise experience in response to an agonist with unbalanced affinity to GLP1R and GCGR. Such reduction also encompasses the elimination of adverse effects previously experienced by a subject following administration of an agonist with unbalanced affinity to GLP1R and GCGR. In some embodiments, “reducing,” or “reduction of” adverse effects encompass a reduction of gastrointestinal side effects wherein the adverse events are reduced to zero or undetectable levels. In other embodiments, adverse effect is reduced to level equivalent to untreated subjects but not completely eliminated. Moreover, administration of analogs with unbalanced affinity toward GLP-1R or GCGR to a mammal may lead to the need for an excessively high dose to maximally activate the receptor with weaker sensitivity toward the ligand, thus leading to a potential for exceeding the biologically effective dose level for the other ligand and causing dose-related, undesired side effects.

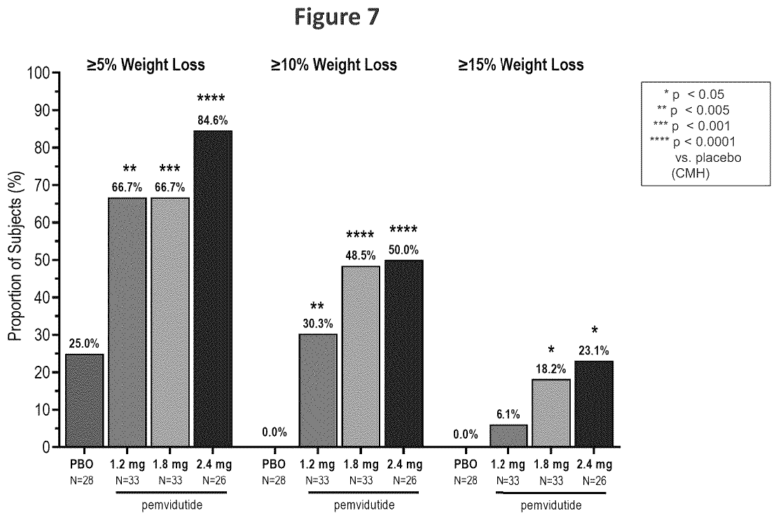

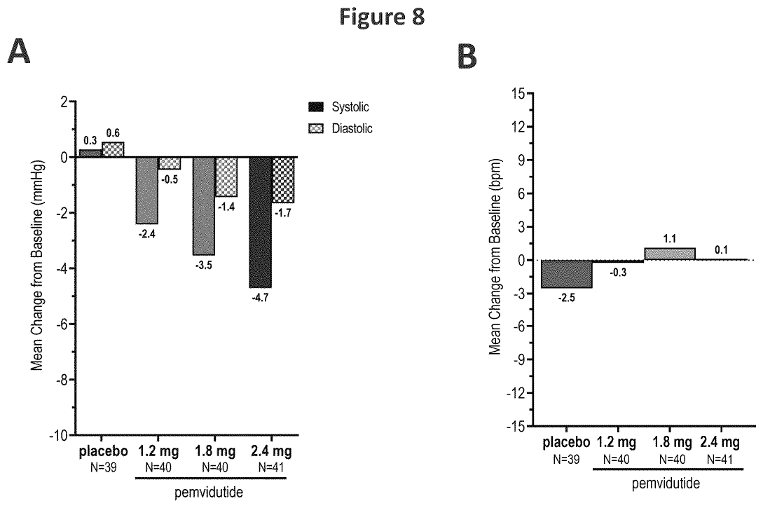

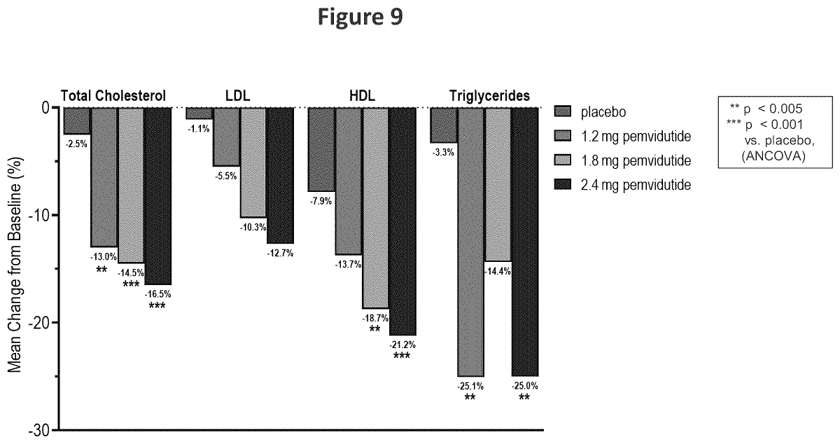

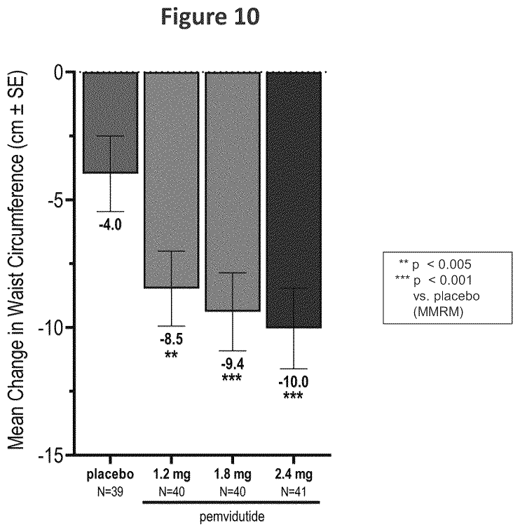

In preferred embodiments, this disclosure provides methods of reducing body weight in a human being with fatty liver disease, wherein the method comprises administering pemvidutide once weekly in an amount from at least 1.8 mg up to 2.4 mg to the human being in need thereof; and, wherein the human being exhibits a CV disease-related conditions (and/or CV disease) and may also be overweight, obese, and/or may also suffer from type 2 diabetes. In some preferred embodiments of such methods, the body weight of the human being is reduced by at least 3% from baseline at week 12. In some preferred embodiments of such methods, the body weight of the human being is reduced by at least 4% from baseline at week 12. In some preferred embodiments of such methods, the pemvidutide is administered once weekly in an amount of 1.8 mg. In some preferred embodiments of such methods, the pemvidutide is administered once weekly in an amount of 2.4 mg. In some preferred embodiments, this disclosure provides methods wherein: about 1.2 mg or about 1.8 mg pemvidutide is administered to a population of human beings for at least 12 weeks wherein, at week 12, the body weight of at least about 65% of the population is reduced by greater than or equal to about 5% from baseline; about 2.4 mg pemvidutide is administered to a population of human beings for at least 12 weeks wherein, at week 12, the body weight of at least about 80% of the population is reduced by greater than or equal to about 5% from baseline; about 1.2 mg pemvidutide is administered to a population of human beings for at least 12 weeks wherein, at week 12, the body weight of at least about 30% of the population is reduced by greater than or equal to about 10% from baseline; about 1.8 or about 2.4 mg pemvidutide is administered to a population of human beings for at least 12 weeks wherein, at week 12, the body weight of at least about 45% of the population is reduced by greater than or equal to about 10% from baseline; about 1.2 mg pemvidutide is administered to a population of human beings for at least 12 weeks wherein, at week 12, the body weight of at least about 5% of the population is reduced by greater than or equal to about 15% from baseline; and/or, about 1.8 or about 2.4 mg pemvidutide is administered to a population of human beings for at least 12 weeks wherein, at week 12, the body weight of at least about 20% of the population is reduced by greater than or equal to about 15% from baseline. See FIG. 7. In some preferred embodiments, the mean change of blood pressure from baseline of the population is decreased. See FIG. 8. In some preferred embodiments, the mean change of blood total cholesterol from baseline of the population is decreased by at least about 10%. See FIG. 9. In some preferred embodiments, the mean change in blood low-density lipoprotein (LDL) from baseline of the population is decreased by at least about 5%. In some preferred embodiments, the mean change in blood high-density lipoprotein (HDL) from baseline of the population is decreased by at least about 15%. In some preferred embodiments, the mean change in blood triglycerides from baseline of the population is decreased by at least about 15%. See FIG. 9. In some preferred embodiments, the mean change in waist circumference of the population is reduced by at least about 8%. See FIG. 10. In some preferred embodiments, the administration of pemvidutide induces a significant increase in ABCG5 (p<0.005) and ABCG8 (p<0.05) gene expression as compared to semaglutide.

In some preferred embodiments, this disclosure provides methods of reducing the risk of cardiovascular (CV) disease in a human being, the method comprising administering pemvidutide once weekly in an amount from at least 1.2 mg up to 2.4 mg to the human being; wherein at least one risk factor associated with CV disease is reduced and/or eliminated, the at least one risk factor being selected from the group consisting of excessive body weight, high serum lipids, cholesterol, triglycerides, LDL and/or VLDL particle concentration and/or diameter, phosphatidylethanolamines, phosphatidylcholines, lysophosphatidylethanolamines, sphingolipids and/or lysophosphatidylcholines in serum of the human being; optionally wherein the human being is overweight, obese, has a body mass index (BMI kg/m²) of greater than or equal to 25, and/or has Type II diabetes. In some preferred embodiments of this method, the pemvidutide is administered once weekly for five to 12 weeks. In some preferred embodiments of this method, the pemvidutide is administered once weekly in an amount of about 1.8 mg; or the pemvidutide is administered once weekly in an amount of about 2.4 mg. In some preferred embodiments of this method, a steady state dose is achieved after a dose escalating phase having a duration of about 2 weeks, about 3 weeks or about 4 weeks; or a steady state dose is achieved after a dose escalating phase having a duration of from two to four weeks. In preferred embodiments of this method, the pemvidutide is administered by parenteral injection; or the pemvidutide is administered by subcutaneous injection. In preferred embodiments of this method, the pemvidutide is administered from a liquid formulation comprising at least about 1.8 mg/ml pemvidutide.

In some preferred embodiments of this method the administration of pemvidutide induces a significant reduction in atherogenic lipids and/or lipoprotein number as compared to baseline. In some preferred embodiments of this method, the atherogenic lipoprotein number concentrations are reduced by at least −0.2 log₂ fold change as compared to baseline following 43 and/or 84 days of administration of pemvidutide; the total serum triglyceride concentration of the subject is reduced by at least −0.2 log₂ fold change relative to placebo following 43 and/or 84 days of administration of pemvidutide; wherein the serum lipids are selected from glycerolipids, sterols, glycerophospholipids, and sphingolipids, optionally wherein the reductions are at least −0.2 log₂ fold change. In some preferred embodiments of this method a reduction in serum phosphatidylethanolamine, phosphatidylcholine, lysophosphatidylethanolamine, sphingolipids and/or lysophosphatidylcholine is induced.

In certain preferred embodiments of this method, the body weight of the human being is reduced by at least 3% or at least 4% from baseline at week 12. In certain preferred embodiments of this method, the human being has type 2 diabetes; and/or the human being has a body mass index (BMI kg/m²) of at least 25, at least about 28, or at least about 30.

In some preferred embodiments, the pemvidutide is administered by parenteral injection. In some preferred embodiments, the pemvidutide is administered by subcutaneous injection. In some preferred embodiments, the human being is overweight, is obese, and/or has a body mass index (BMI kg/m²) of at least 27. In some preferred embodiments, the human being has a body mass index (BMI kg/m²) of 30 or greater. In some preferred embodiments, the human being has level of a parameter related to a CV disease-related condition of 10% or greater before treatment (e.g., at baseline). In some preferred embodiments, the absolute reduction in a CV disease-related condition is any of about 8%, 10%, 12% or preferably about 15% after 12 weeks treatment, as compared to measured values at baseline. In some preferred embodiments, the relative reduction in a CV disease-related condition to baseline is greater than about any of 40%, 50% or 60% after 12 weeks treatment. In some preferred embodiments, a steady state dose is achieved after a dose escalating phase having a duration of two to four weeks, or any of about six, eight, ten, 12, or 16 weeks. In some preferred embodiments, the pemvidutide is administered from a liquid comprising at least about 1.8 mg/ml pemvidutide. In some embodiments, the pemvidutide is administered from a pharmaceutical dosage form as an aqueous formulation comprising one or more of polysorbate 20, Arginine, or Mannitol.

In preferred embodiments, this disclosure relates to the following aspects:
Aspect 1. A method of reducing the risk of cardiovascular (CV) disease in a human being, the method comprising: administering pemvidutide once weekly in an amount from at least 1.2 mg up to 2.4 mg to the human being; wherein at least one risk factor associated with CV disease is reduced and/or eliminated, the at least one risk factor being selected from the group consisting of excessive body weight, high serum lipids, cholesterol, triglycerides, LDL and/or VLDL particle concentration and/or diameter, phosphatidylethanolamines, phosphatidylcholines, lysophosphatidylethanolamines, sphingolipids and/or lysophosphatidylcholines in serum of the human being; optionally wherein the human being is overweight, obese, has a body mass index (BMI kg/m²) of greater than or equal to 25, and/or has Type II diabetes.
Aspect 2. The method of aspect 1 wherein the pemvidutide is administered once weekly in an amount of about 1.2 mg, about 1.8 mg, or about 2.4 mg.
Aspect 3. The method of aspect 1 or 2 wherein the pemvidutide is administered once weekly for five to 12 weeks.
Aspect 4. The method of any one of aspects 1-3, wherein the administration of pemvidutide induces a significant reduction in atherogenic lipids and/or lipoprotein number as compared to baseline.
Aspect 5. The method of any preceding aspect wherein the atherogenic lipoprotein number concentrations are reduced by at least −0.2 log₂ fold change as compared to baseline following 43 and/or 84 days of administration of pemvidutide.
Aspect 6. The method of any preceding aspect wherein the total serum triglyceride concentration of the subject is reduced by at least −0.2 log₂ fold change relative to placebo following 43 and/or 84 days of administration of pemvidutide.
Aspect 7. The method of any preceding aspects wherein the serum lipids are selected from glycerolipids, sterols, glycerophospholipids, and sphingolipids, optionally wherein the reductions are at least −0.2 log₂ fold change.
Aspect 8. The method of any preceding aspect wherein a reduction in serum phosphatidylethanolamine, phosphatidylcholine, lysophosphatidylethanolamine, sphingolipids and/or lysophosphatidylcholine is induced.
Aspect 9. The method of aspect 2, wherein the body weight of the human being is reduced by at least 3% or at least 4% from baseline at week 12.
Aspect 10. The method of any one of any preceding aspect wherein: about 1.2 mg or about 1.8 mg pemvidutide is administered to a population of human beings for at least 12 weeks wherein, at week 12, the body weight of at least about 65% of the population is reduced by greater than or equal to about 5% from baseline; about 2.4 mg pemvidutide is administered to a population of human beings for at least 12 weeks wherein, at week 12, the body weight of at least about 80% of the population is reduced by greater than or equal to about 5% from baseline; about 1.2 mg pemvidutide is administered to a population of human beings for at least 12 weeks wherein, at week 12, the body weight of at least about 30% of the population is reduced by greater than or equal to about 10% from baseline; about 1.8 or about 2.4 mg pemvidutide is administered to a population of human beings for at least 12 weeks wherein, at week 12, the body weight of at least about 45% of the population is reduced by greater than or equal to about 10% from baseline; about 1.2 mg pemvidutide is administered to a population of human beings for at least 12 weeks; wherein, at week 12, the body weight of at least about 5% of the population is reduced by greater than or equal to about 15% from baseline; and/or, about 1.8 or about 2.4 mg pemvidutide is administered to a population of human beings for at least 12 weeks wherein, at week 12, the body weight of at least about 20% of the population is reduced by greater than or equal to about 15% from baseline.
Aspect 11. The method of aspect 10 wherein the mean change of blood pressure from baseline of the population is decreased.
Aspect 12. The method of aspect 10 or 11 wherein the mean change of blood total cholesterol from baseline of the population is decreased by at least about 10%.
Aspect 13. The method of any one of aspects 10-12 wherein the mean change in blood low-density lipoprotein (LDL) from baseline of the population is decreased by at least about 5%.
Aspect 14. The method of any one of aspects 10-13 wherein the mean change in blood high-density lipoprotein (HDL) from baseline of the population is decreased by at least about 15%.
Aspect 15. The method of any one of aspects 10-14 wherein the mean change in blood triglycerides from baseline of the population is decreased by at least about 15%.
Aspect 16. The method of any one of aspects 10-15 wherein the mean change in waist circumference of the population is reduced by at least about 8%.
Aspect 17. The method of any preceding aspect, wherein pemvidutide induces a significant increase in ABCG5 (p<0.005) and ABCG8 (p<0.05) gene expression as compared to baseline.
Aspect 18. The method of any preceding aspect, wherein the pemvidutide is administered once weekly in an amount of about 1.8 mg.
Aspect 19. The method of any preceding aspect, wherein the pemvidutide is administered once weekly in an amount of about 2.4 mg.
Aspect 20. The method of any preceding aspect, wherein a steady state dose is achieved after a dose escalating phase having a duration of about 2 weeks, about 3 weeks or about 4 weeks.
Aspect 21. The method of any preceding aspect, wherein the human being has type 2 diabetes and/or the cardiovascular disease is an atherosclerotic cardiovascular disease.
Aspect 22. The method of any preceding aspect, wherein the human being has a body mass index (BMI kg/m²) of at least 25, at least about 28, or at least about 30.
Aspect 23. The method of any preceding aspect, wherein the pemvidutide is administered by parenteral injection.
Aspect 24. The method of any preceding aspect, wherein the pemvidutide is administered by subcutaneous injection.
Aspect 25. The method of any preceding aspect, wherein a steady state dose is achieved after a dose escalating phase having a duration of from two to four weeks.
Aspect 26. The method of any preceding aspect, wherein the pemvidutide is administered from a liquid formulation comprising at least about 1.8 mg/ml pemvidutide.
Aspect 27. The method of any preceding aspect wherein the pemvidutide is administered as a liquid pharmaceutical formulation comprising SEQ ID NO: 1 and about 0.20% (w/w) polysorbate 20, about 0.348% (w/w) arginine, and about 4.260% (w/w) mannitol in sterile water (pH 7.7±0.1).
Aspect 28. A method of reducing the risk of cardiovascular (CV) disease in a human being, the method comprising: administering pemvidutide once weekly in an amount from at least 1.2 mg up to 2.4 mg to the human being; wherein at least one risk factor associated with CV disease is reduced and/or eliminated, the at least one risk factor being selected from the group consisting of excessive body weight, high serum lipids, cholesterol, triglycerides, LDL and/or VLDL particle concentration and/or diameter, phosphatidylethanolamines, phosphatidylcholines, lysophosphatidylethanolamines, sphingolipids and/or lysophosphatidylcholines in serum of the human being; optionally wherein the human being is overweight, obese, has a body mass index (BMI kg/m²) of greater than or equal to 25, and/or has Type II diabetes.
Aspect 29. A method of treating cardiovascular (CV) disease risk factors comprising: administering a pharmaceutical composition comprising SEQ ID NO: 1, or a pharmaceutically acceptable salt thereof to a patient in need thereof, wherein the patient is overweight, obese, has a body mass index (BMI kg/m²) of greater than or equal to 25, and/or has diabetes mellitus and wherein the method reduces at least one CV disease risk factor as compared to baseline, the CV disease risk factor being selected from the group consisting of body weight, waist circumference, blood pressure, hyperglycemia, serum lipids, total cholesterol, triglycerides, HDL, LDL and/or VLDL particle concentration and/or diameter, phosphatidylethanolamines, phosphatidylcholines, lysophosphatidylethanolamines, sphingolipids and/or lysophosphatidylcholines.
Aspect 30. The method of aspect 29, wherein the patient in need thereof is overweight.
Aspect 31. The method of aspect 29, wherein the patient in need thereof is obese.
Aspect 32. The method of aspect 29, wherein the patient in need thereof has a body mass index (BMI kg/m²) of greater than or equal to 25.
Aspect 33. The method of aspect 29, wherein the patient in need thereof has Type II diabetes.
Aspect 34. The method of aspect 29, wherein the pharmaceutical composition is administered once weekly in an amount of about 1.2 mg, about 1.8 mg, or about 2.4 mg.
Aspect 35. The method of aspect 29, wherein the pharmaceutical composition is administered about once weekly in an amount of about 1.8 mg.
Aspect 36. The method of aspect 29, wherein the pharmaceutical composition is administered about once weekly in an amount of about 2.4 mg.
Aspect 37. The method of aspect 29, wherein a steady state dose is achieved after a dose escalating phase having a duration of about 2 weeks, about 3 weeks or about 4 weeks.
Aspect 38. The method of aspect 29, wherein the pharmaceutical composition is administered once weekly for about five to 12 weeks.
Aspect 39. The method of aspect 29, wherein the pharmaceutical composition is administered by parenteral injection.
Aspect 40. The method of aspect 29, wherein the pharmaceutical composition is administered by subcutaneous injection.
Aspect 41. The method of aspect 29, wherein the pharmaceutical composition is administered from a liquid formulation comprising at least about 1.8 mg/ml pemvidutide.
Aspect 42. The method of any one of aspects 29-41, wherein the pharmaceutical composition is administered as a liquid pharmaceutical formulation comprising SEQ ID NO: 1 and about 0.20% (w/w) polysorbate 20, about 0.348% (w/w) arginine, and about 4.260% (w/w) mannitol in sterilewater (pH 7.7±0.1).
Aspect 43. A method of treating cardiovascular (CV) disease risk factors comprising: administering a pharmaceutical composition comprising SEQ ID NO: 1, or a pharmaceutically acceptable salt thereof to a patient in need thereof, wherein the patient is overweight, obese, has a body mass index (BMI kg/m²) of greater than or equal to 25, and/or has diabetes mellitus and wherein the method reduces at least one of plasma triglycerides, total plasma cholesterol, plasma LDL-cholesterol or plasma HDL-cholesterol levels by about any of 3%, 5% 7%, or preferably 10% from baseline.
Aspect 44. The method of aspect 43, wherein the plasma triglycerides are reduced from baseline.
Aspect 45. The method of aspect 43, wherein the total plasma cholesterol is reduced from baseline.
Aspect 46. The method of aspect 43, wherein the plasma LDL-cholesterol is reduced from baseline.
Aspect 47. The method of aspect 43, wherein the plasma HDL-cholesterol is reduced from baseline.
Aspect 48. A method of reducing plasma lipids in a patient in need thereof, comprising administering a pharmaceutical composition comprising SEQ ID NO: 1, or a pharmaceutically acceptable salt thereof to the patient in need thereof, wherein the patient has established cardiovascular disease and wherein the method reduces at least one of plasma triglycerides, total plasma cholesterol, plasma LDL-cholesterol or plasma HDL-cholesterol levels by 3%, 5%, 7%, or preferably 10% from baseline.
Aspect 49. The method of aspect 48, wherein one or more pathogenic serum lipid mediators are reduced from baseline.

Other aspects of this disclosure are also contemplated as will be understood by those of ordinary skill in the art.

Unless defined otherwise or clearly indicated otherwise by their use herein, all technical and scientific terms used herein have the same meaning as commonly understood by those of ordinary skill in the art to which this application belongs. As used in the specification and the appended claims, the word “a” or “an” means one or more. As used herein, the word “another” means a second or more. The acronym “aka” means also known as. The term “exemplary” as used herein means “serving as an example, instance or illustration”. Any embodiment or feature characterized herein as “exemplary” is not necessarily to be construed as preferred or advantageous over other embodiments or features. In some embodiments, the term “about” or “approximately” means within +10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed. Optional or optionally means that the subsequently described event or circumstance can or cannot occur, and that the description includes instances where the event or circumstance occurs and instances where it does not. All publications, patents, and patent applications mentioned in this specification are herein incorporated by reference in their entireties to the same extent as if each individual publication, patent, or patent application was specifically and individually indicated to be incorporated by reference.

Certain embodiments are further described in the following examples. These embodiments are provided as examples only and are not intended to limit the scope of the claims in any way.

### EXAMPLES

### Example 1. Effect of Pemvidutide (ALT-801), a GLP-1/Glucagon Dual Receptor Agonist, on Pathogenic Lipid Mediators

The potential for CV risk reduction through incretin-based therapy is receiving increased attention. Pemvidutide is a long-acting GLP-1/glucagon (1:1) dual receptor agonist under development for treatment of NASH and obesity. Pemvidutide combines the anorectic effects of GLP-1 receptor agonism (RA) with the increased energy expenditure and lipid-lowering effects of glucagon RA. Plasma lipids serve multiple functions in biological systems such as energy storage, metabolic regulation, signaling, proliferation, and apoptosis. The plasma lipidome can be analyzed using nuclear magnetic resonance (NMR) and ultra-high performance liquid chromatography mass spectrometry (UHPLC-MS).

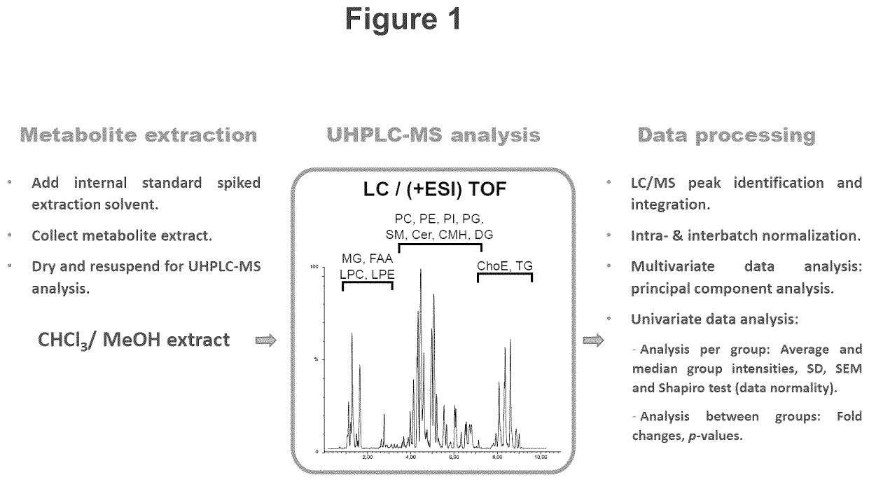

To test the lipid-lowering effects of Pemvidutide, an analysis of Phase I clinical trial (NCT0456124) data was carried out. The subjects with overweight/obesity (BMI 25-40 kg/m²) were randomized at 1 site in Australia (NCT0456124). Subjects were randomized 4:1 pemvidutide:placebo, with placebos pooled. The pemvidutide doses were 1.2 mg, 1.8 mg, and 2.4 mg, administered weekly for twelve (12) weeks, with no dose titration or adjunctive lifestyle intervention (no diet or exercise interventions). Pemvidutide was well-tolerated at all dose levels, without use of dose titration. All AEs in these groups were of mild or moderate severity, so no grade 3 (severe) AEs were seen here and no SAEs or AEs leading to treatment discontinuation were reported. Lipoprotein and glycoprotein profiling covering 33 lipoprotein related parameters was performed by ¹H-NMR on fasting plasma samples obtained at Day −1 (baseline), Day 43, and Day 84 from the 34 subjects who completed NCT0456124. Lipidomic profiling covering 600 lipid species was performed by ultra-high performance liquid chromatography-mass spectrometry on fasting plasma samples obtained at Day −1 (baseline), Day 43, and Day 84 from the 34 subjects who completed NCT0456124. For the lipid profiling, plasma fractionation was performed either using methanol to extract fatty acyls, bile acids, steroids, and lyso-glycerophospholipids, or using a chloroform/methanol mix to extract glycerolipids, cholesteryl esters, sphingolipids, and glycerophospholipids. Lipid classification followed the classification system proposed by Fahy et al. (J. Lipid Res. 2005; 46:839-861) and the LIPID MAPS initiative (http://www.lipidmaps.org. See FIG. 1.

The data presented here shows that pemvidutide had favorable effects on weight loss, body mass index (BMI), blood pressure, total cholesterol. LDL cholesterol, triglycerides, and apoprotein B, as summarized in Table 4:

TABLE 4
Key pharmacodynamic endpoints (change from baseline)

| | | Treatment | | | | | | | |
|---|---|---|---|---|---|---|---|---|---|
| | | 1.2 mg | | 1.8 mg | | 2.4 mg | | Pooled placebo | |
| Endpoint | | (n = 7) | | (n = 9) | | (n = 11) | | (n = 7) | |
| Weight loss, % | mean (SEM) | −4.9 | (1.4) | −10.3 | (1.1)*** | −9.0 | (1.1)** | −1.6 | (1.4) |
| BMI, % | mean (SEM) | −4.8 | (1.22) | −10.4 | (1.14) | −7.0 | (1.12) | −0.8 | (1.37) |
| Systolic BP, % | mean (SEM) | −10.2 | (9.23) | −12.5 | (8.04) | −17.4 | (7.32) | −10.5 | (12.45) |
| Diastolic BP, % | mean (SEM) | −4.7 | (9.73) | −7.1 | (3.02) | −6.0 | (11.47) | −2.7 | (13.54) |
| Total cholesterol, % | mean (SEM) | −20.0 | (2.8) | −28.0 | (3.4)* | −27.7 | (4.6) | −9.1 | (3.7) |
| LDL cholesterol, % | mean (SEM) | −18.4 | (3.6) | −25.6 | (3.7)* | −23.9 | (6.4)* | −4.3 | (6.1) |
| HDL cholesterol, % | mean (SEM) | −16.7 | (1.9) | −30.3 | (3.2) | −36.0 | (3.8) | −19.2 | (5.7) |
| Triglycerides, % | mean (SEM) | −37.0 | (1.7)** | −37.9 | (7.7)*** | −29.2 | (7.7)* | −8.2 | (7.0) |
| Apoprotein B, % | mean (SEM) | −45.4 | (4.8) | −23.4 | (3.3) | −16.8 | (10.9) | −9.9 | (8.0) |

*p < 0.05,
**p < 0.01,
***p < 0.001, vs. placebo

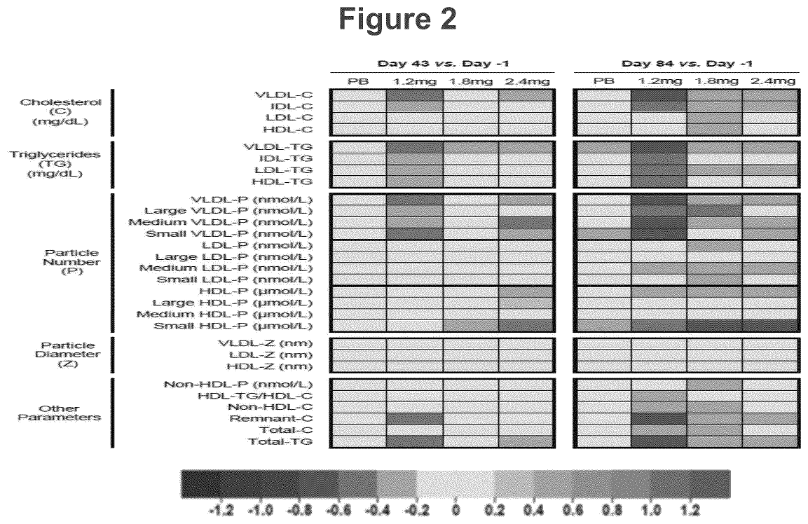

Pemvidutide also caused generally consistent changes in lipoprotein particle subspecies as determined by 2D-NMR analysis. See FIG. 2. The color code represents the log 2 (robust fold-change) with blue colors denoting reduced lipoproteins (negative fold-changes) and red colors denoting increased lipoproteins (positive fold-changes). FIG. 2 shows that the number of VLDL and LDL particles and the number of smaller HDL particles tend to be reduced following treatment with pemvidutide.

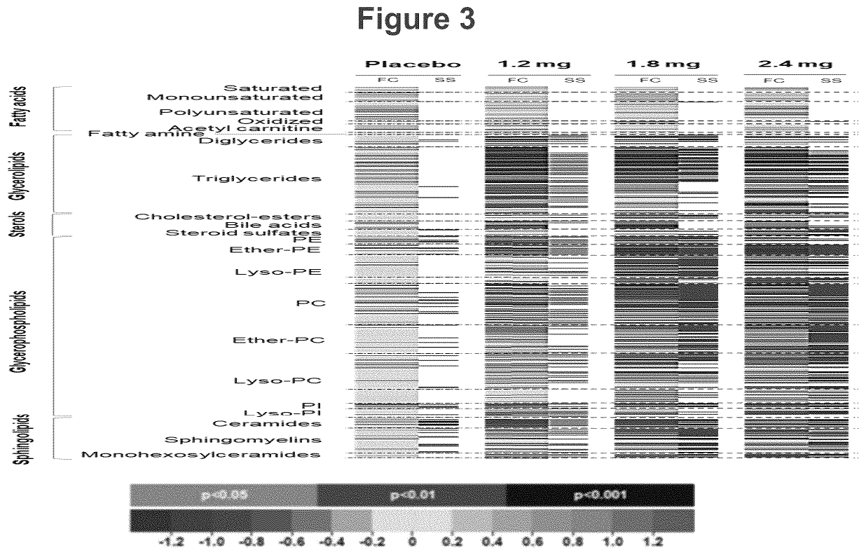

Given the observed changes in the total cholesterol, triglycerides and lipoproteins, an evaluation of the serum lipid composition covering 600 lipid species at Day −1 (baseline), Day 43, and Day 84 from the 34 subjects who completed NCT0456124 was performed. Results are presented in FIG. 3 as log 2 pairwise fold change with blue colors denoting a reduction in the lipid relative to baseline, and red an increase. The grey/black bars indicate significant p-values of Wilcoxon test. Within 12 weeks of treatment, pemvidutide significantly reduced serum lipid levels, especially glycerolipids (diglycerides and triglycerides), glycerophospholipids (phosphatidylethanolamines, phosphatidylcholines, lysophosphatidylethanolamines, and lysophosphatidylcholines) and sphingolipids (ceramides and sphingomyelins) associated with reduction in cardiovascular (CV) risk as well as reduction in insulin resistance. See FIG. 3. Thus, at Day 85, substantial and highly statistically significant reductions across multiple bioactive lipid classes were observed compared to placebo.

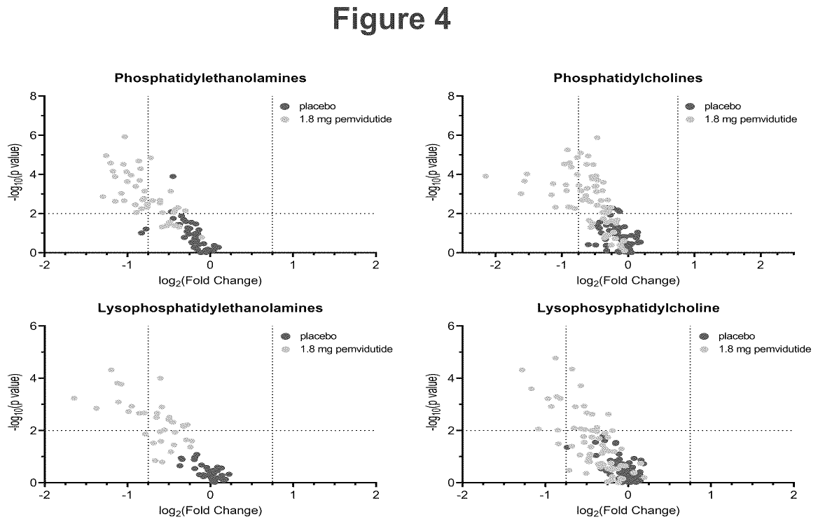

Based on this lipodomics data, volcano plots were generated as shown in FIG. 4, focusing on changes in atherogenic lipids following treatment with 1.8 mg pemvidutide. The horizontal dashed line represents the lower limit of significance, while the vertical lines show a fold-change from baseline of ±0.75 on the log 2 scale. Responses in the upper left quadrant indicate particularly meaningful changes in the atherogenic lipids. Pemvidutide treatment significantly reduced pro-atherogenic lyso-PC levels (See FIG. 4), suggesting a potential for decreased oxidized LDL (Law et al. Int. J. Mol. Sci. 2019; 20(5):1149). Pemvidutide treatment also significantly reduced atherosclerotic plaque forming phosphatidyl-ethanolamine (PE) glycerophospholipids level. Based on these findings, pemvidutide demonstrates promise as an agent for reducing cardiovascular (CV) risk.

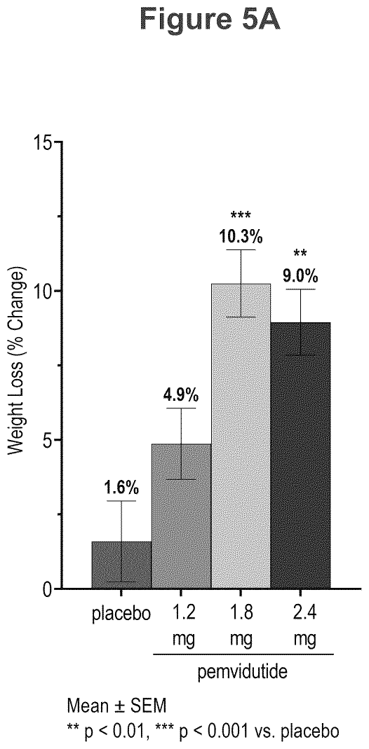

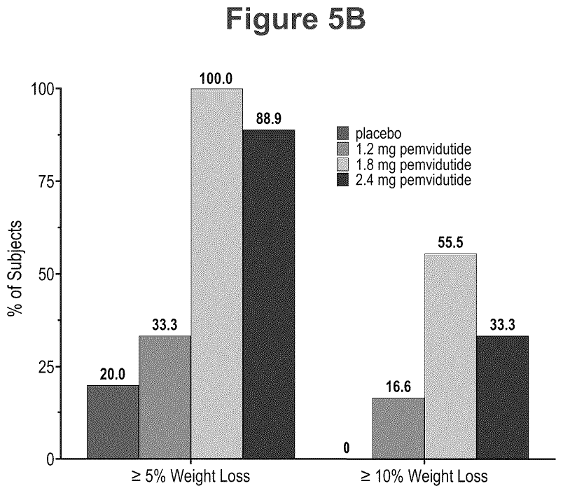

In this trial, pemvidutide induced substantial weight loss at week 12 as illustrated in FIGS. 5A and 5B. Body weight losses of up to a mean of 10.3% (8.7% after subtraction of placebo) at the 1.8 mg dose level were observed, with a high degree of statistical significance after just 12 weeks of weekly treatment. Additionally, within this treatment group all subjects lost at least 5% of their body weight and over half achieved at least 10% body weight loss.

Pemvidutide was shown safe and well-tolerated, without dose-titration in this Phase 1a Trial. In this study, up to 10.3% mean weight losses were observed, with statistically significant reductions across multiple atherogenic lipid classes as well as in atherogenic lipoprotein particles. Based on these findings, pemvidutide demonstrates promise as an agent for reducing risk for CV disease.

### Example 2: Efficacy on Pathogenic Lipid Mediators of ALT-801 (Pemvidutide) in a Phase II, 24-Week, Randomized, Double-Blind, Placebo-Controlled Study with Non-Diabetic Obese and Overweight Subjects

Results presented in this example correspond to a 24-week interim analysis obtained from a placebo-controlled phase II study (ClinicalTrials.gov Identifier: NCT05295875) evaluating pemvidutide (ALT-801) in obese and overweight subjects. Key eligibility criteria were: (1) Men and women, ages 18-75 years; (2) at least one unsuccessful weight loss attempt per Investigator judgement; (3) Body Mass Index (BMI)≥30 kg/m² or BMI≥27 kg/m² with at least one obesity-related comorbidity (history of cardiovascular disease, hypertension, dyslipidemia, pre-diabetes or obstructive sleep apnea) and (4) non-diabetes (HbAlc≤6.5% and fasting glucose≤125 mg/dL). Eligible subjects were randomized 1:1:1:1 to one of the following treatment arms: Group 1 (39 subjects): pemvidutide 1.2 mg SC once weekly for 24 weeks; Group 2 (40 subjects): pemvidutide 1.8 mg administered subcutaneously once weekly for 24 weeks; Group 3 (40 subjects): pemvidutide 0.6 mg administered subcutaneously for 1 week, 1.2 mg administered subcutaneously for 1 week, 1.8 mg administered subcutaneously once weekly for 2 weeks, followed by 2.4 mg administered subcutaneously once weekly for 20 additional weeks (in sequence) and Group 4 (41 subjects): Placebo administered subcutaneously once weekly for 24 weeks. The randomization of subjects was stratified based on sex and baseline body mass index (BMI<35 kg/m² vs. ≥35 kg/m²). A minimum of 25% of randomized subjects were male. All subjects received counselling on a reduced-calorie diet of 1200-1500 calories for individuals <250 lbs (113.6 kg) and 1500-1800 calories for individuals ≥250 lbs (113.6 kg) and a gradual increase in physical activity (targeting 150 min of physical activity per week) by a qualified healthcare professional at screening and at subsequent visits during the treatment period. Subjects were instructed to record their food intake and physical activity daily, and compliance with lifestyle interventions will be assessed by the investigator on a regular basis. A subgroup of subjects was subjected to MRI-PDFF to evaluate the hepatic fat fraction in as well as composition to measure total body adipose tissue (AT) and adipose tissue-free mass (ATFM).

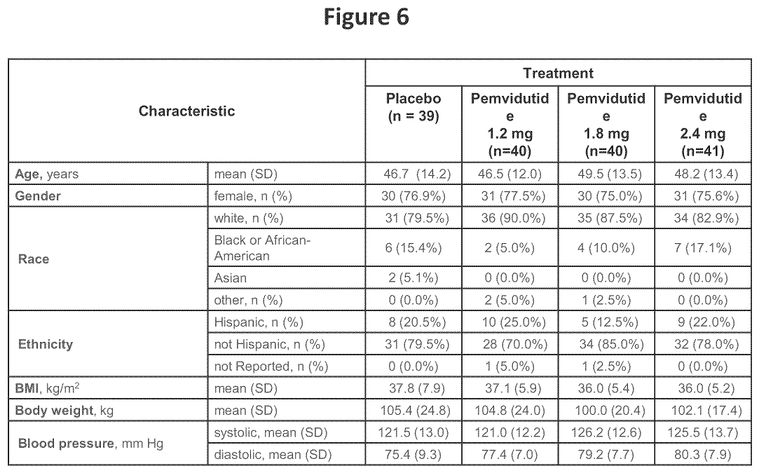

Baseline characteristics of study participants are presented in FIG. 6. FIG. 7 presents a weight loss responder analysis based on calculation of percentage of subjects who respectively achieve ≥5%, ≥10% and ≥15% weight loss across the three pemvidutide doses and placebo. FIG. 8A shows that 24-week treatment with pemvidutide improves both systolic and diastolic blood pressures in a dose dependent manner as opposed to placebo. This result is important considering that obesity represents a major cause of hypertension. FIG. 8B shows that 24-week treatment with pemvidutide at 1.2 mg, 1.8 mg and 2.4 mg doses does not induce a significant change in heart rates. FIG. 9 shows serum lipids at week 24 as compared to baseline for all doses of pemvidutide administered (1.2 mg, 1.8 mg, and 2.4 mg). FIG. 9 shows improvements in serum lipids at week 24. As shown therein, e.g., improvements in serum lipids for all doses of pemvidutide administered (1.2 mg, 1.8 mg, and 2.4 mg per week) were observed. FIG. 10 shows a significant reduction in waist circumference at week 24 for all doses of pemvidutide administered (1.2 mg, 1.8 mg, and 2.4 mg per week). Waist circumference is an index of central or abdominal obesity recommended by the WHO to assess the risk of metabolic diseases such as NAFLD and NASH and cardiovascular diseases.

### Example 3: Effects of Pemvidutide on Macrophage-to-Feces Reverse Cholesterol Transport in Diet-Induced Obese Hamsters

The study was conducted in male Golden Syrian hamsters placed on a free choice diet (free choice between a chow diet with normal tap water or a high fat/cholesterol diet (Safe Diets, 40.8% fat, 14.8% protein, 44.4% carbohydrates and 0.5% cholesterol, 4444.2 kcal/kg diet) with 10% fructose enriched tap water (0.4 kcal/L) provided ad libitum for a minimum of 20 weeks prior to the study start and until the end of the experiment. After randomization based on body weight and plasma cholesterol levels, hamsters were assigned to two groups of ten (10) animals receiving vehicle or pemvidutide (10 nmol/kg) administered subcutaneously daily for 35 days and during a 72-hour macrophage-to-feces reverse cholesterol transport (RCT) experiment. Body weight was measured three (3) times per week. On day 35, hamsters were six (6)-hour fasted and blood (150 μL/heparin) was collected by retro-orbital bleeding under isoflurane anesthesia to isolate plasma and measure fasting plasma triglycerides, total cholesterol, LDL-cholesterol and HDL-cholesterol levels. On day 36, Hamsters were injected intraperitoneally with oxidized LDL-loaded/[3H]-cholesterol labeled J774 macrophages to perform a 72-hour macrophage-to-feces RCT experiment, as previously described in Castro-Perez et al (Castro-Perez, et al.. Anacetrapib promotes reverse cholesterol transport and bulk cholesterol excretion in Syrian golden hamsters. J Lipid Res. 2011; 52: 1965-73). Blood was collected on heparin tubes, under slight isoflurane anesthesia 24, 48 and 72 hours after injection of the labeled J774 cells to measure radioactivity in plasma. Feces were continuously collected during the 72-hours experiment then weighed prior to measuring fecal [3H]-cholesterol and [3H]-bile acids after chemical extraction from feces homogenate. After 72 hours, hamsters were sacrificed, and livers were collected to measure hepatic lipid after chemical extraction from liver sample homogenate.

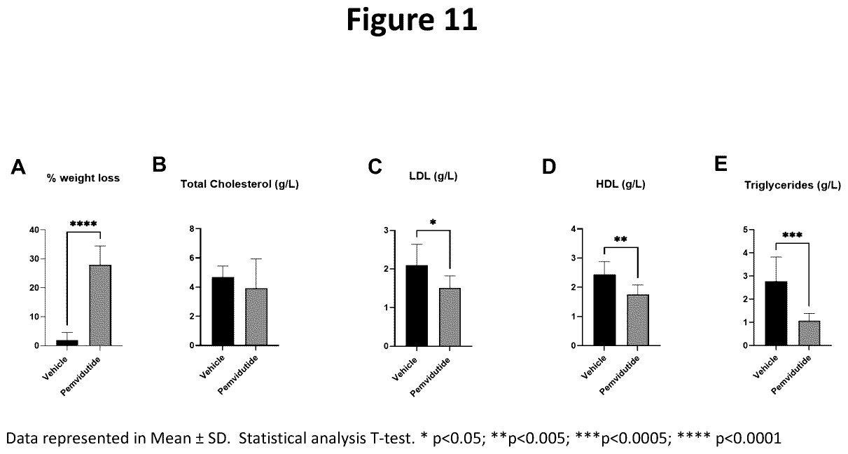

FIG. 11A shows the significant impact of pemvidutide treatment on the percentage change in body weight from baseline (p<0.0001) compared to the vehicle group at day 35. FIG. 11B to 11E show the significant effect of pemvidutide treatment on lowering plasma lipids including LDL-c (p<0.05), HDL-c (p<0.005) and triglycerides (p<0.0005) compared to vehicle at day 35. Lower plasma cholesterol was also observed resulting from pemvidutide treatment days as compared to vehicle, but this difference was not considered statistically significant. Both the reduction in body weight and plasma lipids are aligned with the intended mechanism of action of pemvidutide and broadly replicate the activity of the drug observed in clinical studies as presented in Example 1 and Example 2.

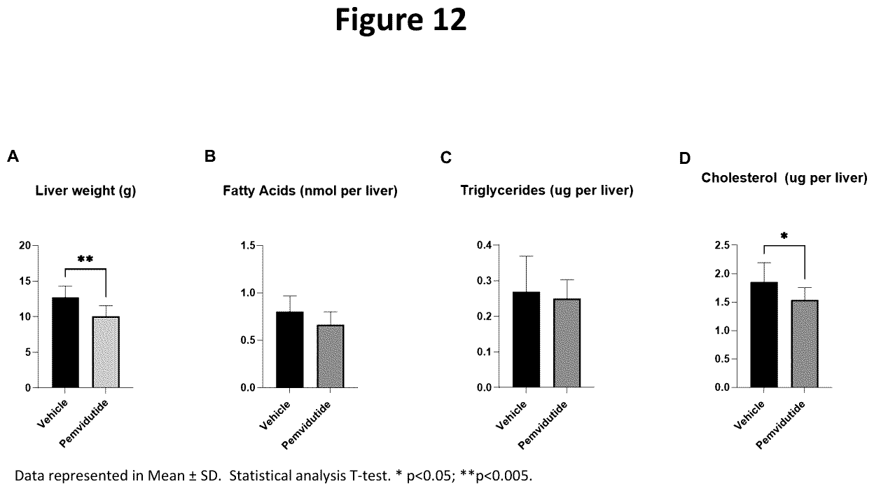

FIG. 12A shows the significant impact of pemvidutide treatment on the reduction in liver weight (p<0.005) compared to the vehicle group. In addition, FIGS. 12B, 12C and 12D respectively show the impact of pemvidutide treatment for 35 days on lowering cholesterol (p<0.05), triglyceride and fatty acids compared to vehicle.

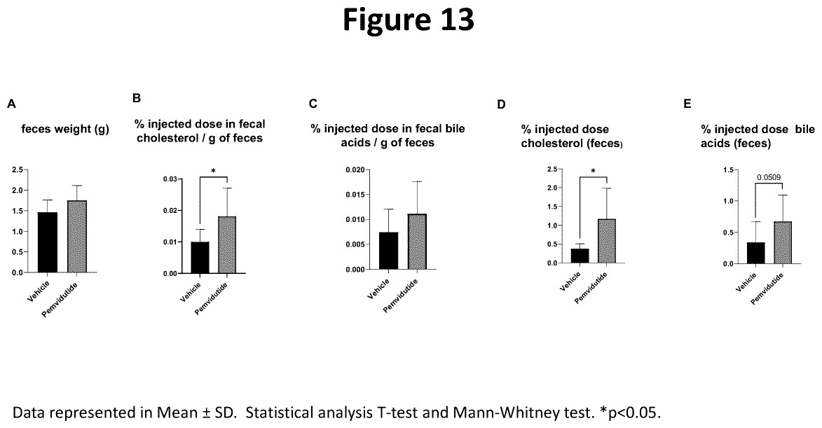

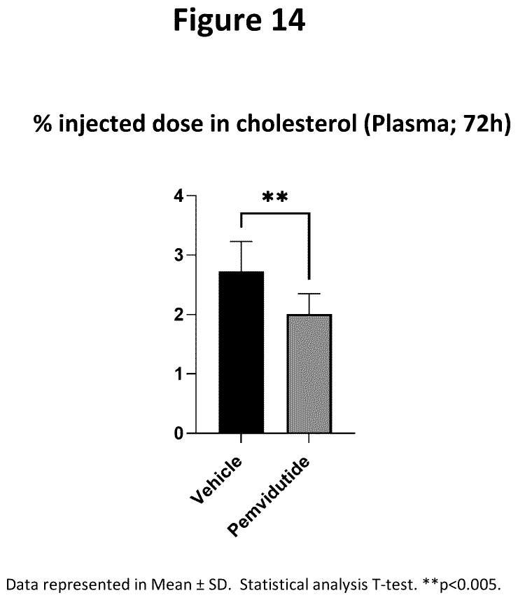

Following injection of the [3H]-cholesterol labeled J774 macrophages, [3H]-cholesterol and/or [3H]-bile acid (resulting from the enzymatic conversion of [3H]-cholesterol in the liver) were measured in plasma, liver and feces samples at 72 hours, and expressed as the percentage of [3H]-cholesterol injected dose. Taking into consideration the equivalent amount of feces output across the two groups (FIG. 13A), FIGS. 13B to 13E show that pemvidutide promotes a significant increase in [3H]-cholesterol (p<0.05) and a trend toward an increase in [3H]-bile acid in the feces, expressed as the % of injected dose per gram of feces or total feces weight, compared to vehicle. Related to this result, FIG. 14 shows the effect of pemvidutide treatment on lowering [3H]-cholesterol in plasma compared to vehicle group, possibly reflecting a more efficient cholesterol transport from the peripheral labeled macrophages to the liver.

Taken together, these results support the beneficial impact of pemvidutide on reverse cholesterol transport (RCT), a mechanism associated with an increased removal of cholesterol from peripheral macrophages and elimination in the feces. This result can also be related to the reduction in plasma HDL-c levels observed with pemvidutide, possibly reflecting a higher turnover of these lipid particles known to mediate RCT. In conclusion, pemvidutide promotes RCT, an anti-atherosclerotic mechanism associated with cardiovascular benefits in addition to weight loss.

### Example 4: Effects of Pemvidutide on Gene Expression Involved in Reverse Cholesterol Transport in a Diet-Induced Obese Hamster Model

The study was conducted in male Golden Syrian hamsters placed on a free choice diet (free choice between a chow diet with normal tap water or a high fat/cholesterol diet (Safe Diets, 40.8% fat, 14.8% protein, 44.4% carbohydrates and 0.5% cholesterol, 4444.2 kcal/kg diet) with 10% fructose enriched tap water (0.4 kcal/L) provided ad libitum for a minimum of 20 weeks prior to the study start and until the end of the experiment. After randomization based on body weight, hamsters were assigned to three groups of 5 animals receiving vehicle, pemvidutide (10 nmol/kg) or semaglutide (10 nmol/kg) administered subcutaneously daily for 21 days. After 21 days, hamsters were sacrificed, and livers were collected. Total RNA was extracted from frozen liver tissue before measuring gene expression by RT-PCR across a range of markers including CYP7A, ABCA1, ABCG1, ABCG5, ABCG8, SREBP1c, SRB1 and LDL-R.

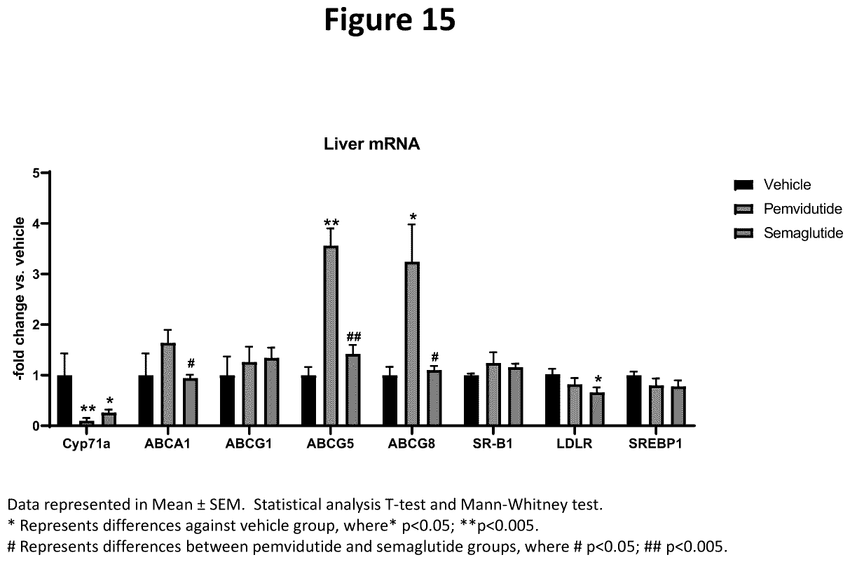

FIG. 15 shows the effect of pemvidutide on the liver expression of CYP7A, ABCA1, ABCG1, ABCG5, ABCG8, ACAT2, SREBP1c, SRB1 or LDL-R compared to semaglutide or vehicle. Results are expressed as fold change compared to vehicle. Surprisingly, pemvidutide induces a significant increase in ABCG5 and ABCG8 gene expression as opposed to Semaglutide and vehicle. This results support the effect of pemvidutide on ABCG5 and ABCG8 genes as a result of its glucagon activity in the liver, an organ that expressed the receptor for glucagon and not the receptor for GLP-1. Hepatic ATP-binding cassette (ABC) transporters G5 (ABCG5) and G8 (ABCG8) are both known to facilitate biliary secretion of cholesterol and phytosterols into the intestine. Mutations in either of the two genes cause sitosterolemia, a condition in which cholesterol and plant sterols accumulate in the circulation leading to premature cardiovascular disease (Tada, et al. Adv Clin Chem. 2022; 110:145-169). Overexpression of ABCG5 and ABCG8 in mice retards diet-induced atherosclerosis because of reduced circulating and hepatic cholesterol (Wilund, et al. High-level expression of ABCG5 and ABCG8 attenuates diet-induced hypercholesterolemia and atherosclerosis in Ldlr−/− mice. J Lipid Res 45: 1429-1436, 2004). Overall, this result indicates that pemvidutide may exert an anti-atherosclerotic role through the overexpression of ABCG5 and ABCG8 in the liver as a mechanism supporting reverse cholesterol transport.

Accordingly, provided herein is a method of using pemvidutide for reducing the risk of cardiovascular (CV) disease in a non-diabetic overweight or obese human being, wherein the method comprises administering pemvidutide once weekly in an amount from at least 1.2 mg up to 2.4 mg to the human being; wherein at least one risk factor associated with CV disease is reduced and/or eliminated, the at least one risk factor being selected from the group consisting of excessive body weight, cholesterol, triglycerides, HDL and LDL in serum of the human being.

Other advantages of the reagents and methods of using the same are also provided herein, as would be understood by those of ordinary skill in the art. While certain embodiments have been described in terms of the preferred embodiments, it is understood that variations and modifications will occur to those skilled in the art. Therefore, it is intended that the appended claims cover all such equivalent variations that come within the scope of the following claims.

1-28. (canceled)

29. A method of treating cardiovascular (CV) disease risk factors comprising:
administering a pharmaceutical composition comprising a therapeutically effective amount of SEQ ID NO: 1, or a pharmaceutically acceptable salt thereof to a patient in need thereof, wherein the patient is overweight, obese, has a body mass index (BMI kg/m²) of greater than or equal to 25, and/or has Type II diabetes and wherein the method reduces at least one CV disease risk factor as compared to baseline, the CV disease risk factor being selected from the group consisting of body weight, waist circumference, blood pressure, hyperglycemia, serum lipids, total cholesterol, triglycerides, HDL, LDL and/or VLDL particle concentration and/or diameter, phosphatidylethanolamines, phosphatidylcholines, lysophosphatidylethanolamines, sphingolipids and/or lysophosphatidylcholines.

30. The method of claim 29, wherein the patient in need thereof is overweight.

31. The method of claim 29, wherein the patient in need thereof is obese.

32. The method of claim 29, wherein the patient in need thereof has a body mass index (BMI kg/m²) of greater than or equal to 25.

33. The method of claim 29, wherein the patient in need thereof has Type II diabetes.

34. The method of claim 29, wherein the pharmaceutical composition is administered once weekly in an amount of about 1.2 mg, about 1.8 mg, or about 2.4 mg.

35. The method of claim 29, wherein the pharmaceutical composition is administered about once weekly in an amount of about 1.8 mg.

36. The method of claim 29, wherein the pharmaceutical composition is administered about once weekly in an amount of about 2.4 mg.

37. The method of claim 29, wherein a steady state dose is achieved after a dose escalating phase having a duration of about 2 weeks, about 3 weeks or about 4 weeks.

38. The method of claim 29, wherein the pharmaceutical composition is administered once weekly for about five to 12 weeks.

39. The method of claim 29, wherein the pharmaceutical composition is administered by parenteral injection.

40. (canceled)

41. The method of claim 29, wherein the pharmaceutical composition is administered from a liquid formulation comprising at least about 1.8 mg/ml pemvidutide.

42. The method of claim 29, wherein the pharmaceutical composition is administered as a liquid pharmaceutical formulation comprising SEQ ID NO: 1 and about 0.20% (w/w) polysorbate 20, about 0.348% (w/w) arginine, and about 4.260% (w/w) mannitol in sterile water (pH 7.7±0.1).

43. A method of treating cardiovascular (CV) disease risk factors comprising:
administering a pharmaceutical composition comprising a therapeutically effective amount of SEQ ID NO: 1, or a pharmaceutically acceptable salt thereof to a patient in need thereof, wherein the patient is overweight, obese, has a body mass index (BMI kg/m²) of greater than or equal to 25, and/or has Type II diabetes and wherein the method reduces at least one of plasma triglycerides, total plasma cholesterol, plasma LDL-cholesterol or plasma HDL-cholesterol levels by about any of 3%, 5% 7%, or 10% from baseline.

44. The method of claim 43, wherein the plasma triglycerides are reduced from baseline.

45. The method of claim 43, wherein the total plasma cholesterol is reduced from baseline.

46. The method of claim 43, wherein the plasma LDL-cholesterol is reduced from baseline.

47. The method of claim 43, wherein the plasma HDL-cholesterol is reduced from baseline.

48. A method of reducing plasma lipids in a patient in need thereof, comprising administering a pharmaceutical composition comprising a therapeutically effective amount of SEQ ID NO: 1, or a pharmaceutically acceptable salt thereof to the patient in need thereof, wherein the patient has established cardiovascular disease and wherein the method reduces at least one of plasma triglycerides, total plasma cholesterol, plasma LDL-cholesterol or plasma HDL-cholesterol levels by 3%, 5%, 7%, or 10% from baseline.

49-60. (canceled)

61. The method of claim 29, wherein the pharmaceutical composition is administered as a liquid pharmaceutical formulation comprising SEQ ID NO: 1 and about 0.20% (w/w) polysorbate 20, about 0.348% (w/w) arginine, and about 4.260% (w/w) mannitol in sterile water (pH 7.7±0.1).
